(19) United States
(12) Patent Application Publication
(10) Pub. No.: US 2026/0080975 A1
(43) Pub. Date: Mar. 19, 2026

(54) METHODS AND SYSTEMS FOR PREDICTING A DISEASE STATE BASED ON ANALYZING CFDNA FRAGMENTS

(71) Applicant: Foundation Medicine, Inc., Boston, MA (US)

(72) Inventors: Mark R. KENNEDY, Cambridge, MA (US); Justin NEWBERG, Cambridge, MA (US)

(73) Assignee: Foundation Medicine, Inc., Boston, MA (US)

(21) Appl. No.: 19/313,294

(22) Filed: Aug. 28, 2025

Related U.S. Application Data
(60) Provisional application No. 63/696,011, filed on Sep. 18, 2024.

(51) Int. Cl. G16B 30/10; A61K 45/06; C12Q 1/6855; C12Q 1/6869; G16H 20/10; G16H 50/20

(52) U.S. Cl. CPC G16B 30/10; A61K 45/06; C12Q 1/6855; C12Q 1/6869; G16H 20/10; G16H 50/20; C12Q 2600/154

(57) ABSTRACT

Methods for predicting a disease based on analyzing cfDNA fragments are described. The methods may comprise, for example, determining, by one or more processors, a test count of methylated loci for cfDNA fragments within a size range for a test sample obtained from a subject; determining, by the one or more processors, a disease score based on comparing the test count of the methylated loci to a reference count of methylated loci from one or more reference samples; and predicting, by the one or more processors, the disease state for the subject based on a comparison of the disease score to a predetermined disease score threshold.

### CROSS-REFERENCE TO RELATED APPLICATIONS

This application claims priority to U.S. Provisional Patent Application No. 63/696,011, filed on Sep. 18, 2024, the disclosure of which is hereby incorporated by reference in its entirety.

### FIELD

The present disclosure relates generally to methods and systems for predicting a disease state, and more specifically to methods and systems for predicting a disease state based on analyzing features of cell-free DNA (cfDNA) fragments.

### BACKGROUND

Cell-free DNA (cfDNA) fragments can be used to predict a subject's disease state, e.g., the likelihood that the individual has or will eventually manifest a disease or a disease score. Extracting cfDNA fragments is relatively non-invasive compared to other methods of DNA extraction and often times there is not tissue that can be used to assess disease. For this reason, analyzing cfDNA fragment sequences provides a promising approach by which to predict the disease state for a subject. However, not all cfDNA may be relevant to disease analysis. For some diseases like cancer, cfDNA from the tumor is the most clinically relevant. This type of cfDNA is called circulating tumor DNA (ctDNA). A key difficulty in analyzing ctDNA in the sea of cfDNA fragments is how to distinguish ctDNA from cfDNA. ctDNA fragments, especially at early stages of disease, can be present at low levels in a subject, relative to other types of DNA molecules. Accordingly, extracting reliable clinically relevant information from cfDNA can be challenging.

Existing methods have attempted to extract information from cfDNA sequences and identify it as ctDNA by analyzing a singular feature of cfDNA molecules. For example, some existing methods aim to predict a subject's disease state by analyzing only the methylation status of cfDNA fragments. Other existing methods aim to predict a subject's disease state by analyzing only the sequence variants of cfDNA fragments. The analyzing of a single feature for cfDNA fragments can, however, often be error-prone, limited, and unreliable, especially when the signal is so weak. Existing methods rarely integrate methylation, fragment characteristics (e.g., length and topography), and sequences of DNA molecules for disease prediction (Cescon et al., Circulating tumor DNA and liquid biopsy in oncology, Nature Cancer. 1:276-290. (2020)). In the cases where multiple features of cfDNA fragments are, in fact, analyzed together, computationally intense and heuristic processes are often used, which can be difficult for a clinician to interpret (Kim et al., Cancer signature ensemble integrating cfDNA methylation, copy number, and fragmentation facilitates multi-cancer early detection, Experimental and Molecular Medicine. 55:2445-2460. (2023). The current methods for analyzing cfDNA fragments can be improved upon, both in terms of reliability and computational efficiency. The methods and systems disclosed herein aim to address these issues.

### BRIEF SUMMARY OF THE INVENTION

Disclosed herein are methods and systems for predicting a disease state based on analyzing features of cell-free (cfDNA) fragments, such as methylation status and size. Existing methods of predicting disease states are oftentimes limited to analyzing only a single feature of the cfDNA fragments, e.g., the sequence. Such a limited analysis, however, can curtail the method's ability to accurately predict the disease. The methods and systems described herein integrate multiple features of cfDNA fragments, to predict a disease state in a subject. The integration of multiple features allows for improved predictive accuracy for not only multiple cancer types, but for multiple stages of the cancer types, e.g., stage II lung cancer. The methods and systems described herein comprise analyzing both the methylation status, as well as the size, e.g., base pair length, of cfDNA fragments. More specifically, the analysis can include stratifying the cfDNA fragments by size, and then comparing the methylated loci for a given size range, e.g., a test count of methylated loci, to different methylated loci from another sample that are also of the same size range, e.g., a reference count of methylated loci from one or more reference samples. From the comparison, a disease score can be determined, and if the disease score is greater or less than a predetermined disease score threshold, the disease state (e.g., stage) can be predicted for the subject, e.g., a likelihood for the predicted disease state can be determined for the subject. By analyzing, for example, both the methylation patterns and the size of cfDNA fragments, the methods described herein allow for highly accurate disease predictions at a computationally efficient cost that is easily interpretable.

In some aspects, disclosed herein is a method comprising: providing a plurality of cfDNA fragments obtained from a sample from a subject; ligating one or more adapters onto one or more cfDNA fragments from the plurality of cfDNA fragments; amplifying the one or more ligated cfDNA fragments from the plurality of cfDNA fragments; capturing amplified cfDNA fragments from the amplified cfDNA fragments; sequencing, by a sequencer, the captured cfDNA fragments to obtain a plurality of sequence reads that represent the captured cfDNA fragments; receiving, by one or more processors, sequence read data for the plurality of sequence reads; aligning, by the one or more processors, the sequence read data to a reference genome, thereby generating computationally reconstructed cfDNA fragments (CRCFs); determining, by the one or more processors, a test count of methylated loci for a CRCF of the CRCFs, within a size range, for a test sample obtained for the subject; determining, by the one or more processors, a disease score based on comparing the test count of the methylated loci to a reference count of methylated loci from one or more reference samples; and predicting, by the one or more processors, the disease state for the subject based on a comparison of the disease score to a predetermined disease score threshold.

In some aspects, disclosed herein is a method further comprising: binning, by the one or more processors, sizes of the CRCFs; and determining, by the one or more processors, the size range based on the binned sizes. In any of the embodiments herein, disclosed herein is a method further comprising: preprocessing one or more methylation statuses of one or more nucleotides from one or more ends of sequence reads from the CRCFs. In some embodiments, the preprocessing can be based on one or more methylation statuses of other sequence reads from the CRCFs. In any of the embodiments herein, the preprocessing can comprise computationally removing the one or more nucleotides from the one or more ends of the sequence reads. In any of the embodiments herein, the preprocessing can comprise computationally correcting the one or more methylation statuses of the one or more nucleotides. In any of the embodiments herein, the disease state can indicate being at risk or having colorectal cancer (CRC). In some embodiments, the colorectal cancer can be stage 1 CRC, stage II CRC, stage III CRC, or stage IV CRC.

In any of the embodiments herein, the disease state can indicate being at risk or having lung cancer. In any of the embodiments herein, the lung cancer can be stage I lung cancer, stage II lung cancer, stage III lung cancer, or stage IV lung cancer.

In any of the embodiments herein, the disease score can be a probability from binomial testing, or a Kullback-Leibler divergence. In some embodiments, the probability from the binomial testing can be based on the determined test count of the methylated loci and the reference count of methylated loci. In any of the embodiments herein, the subject can be suspected of having or is determined to have cancer.

In any of the embodiments herein, the CRCFs can be based on an aligning of sequence reads from the same fragment against a reference genome, thereby generating one or more merged reads. In some embodiments, the CRCFs from the same molecule can be grouped together to generate one or more consensus sequences.

In some embodiments, the cancer can be a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer can comprise acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some aspects, disclosed herein is a method further comprising obtaining the test sample from the subject. In any of the embodiments herein, the test sample can comprise a liquid biopsy sample, or a normal control. In some embodiments, the test sample can be a liquid biopsy sample and comprises blood, plasma, cerebrospinal fluid, sputum, stool, urine, or saliva.

In some embodiments, the test sample can be a liquid biopsy sample and comprises circulating tumor cells (CTCs).

In some embodiments, the test sample can be a liquid biopsy sample and comprises cell-free DNA (cfDNA). In some embodiments, the cell-free DNA (cfDNA) or a portion thereof comprises circulating tumor DNA (ctDNA). In any of the embodiments herein, the plurality of cfDNA fragments comprises a mixture of tumor cfDNA fragments and non-tumor cfDNA fragments. In some embodiments, the tumor cfDNA fragments can be derived from a tumor portion of a heterogeneous liquid biopsy sample, and the non-tumor cfDNA fragments are derived from a normal portion of the heterogeneous liquid biopsy sample. In some embodiments, the test sample can comprise a liquid biopsy sample, and wherein the tumor cfDNA fragments are derived from a circulating tumor DNA (ctDNA) fraction of the liquid biopsy sample, and the non-tumor cfDNA fragments are derived from a non-tumor, cell-free DNA (cfDNA) fraction of the liquid biopsy sample. In any of the embodiments herein, the one or more adapters can comprise amplification primers, flow cell adaptor sequences, substrate adapter sequences, or sample index sequences. In any of the embodiments herein, the captured cfDNA fragments can be captured from the amplified cfDNA fragments by hybridization to one or more bait molecules.

In some embodiments, the one or more bait molecules can comprise one or more cfDNA fragments, each comprising a region that is complementary to a region of a captured cfDNA fragment. In any of the embodiments herein, amplifying cfDNA fragments can comprise performing a polymerase chain reaction (PCR) amplification technique, a non-PCR amplification technique, or an isothermal amplification technique. In any of the embodiments herein, the sequencing can comprise use of a massively parallel sequencing (MPS) technique, whole genome sequencing (WGS), whole exome sequencing, targeted sequencing, direct sequencing, or Sanger sequencing technique. In some embodiments, the sequencing can comprise massively parallel sequencing, and the massively parallel sequencing technique comprises next generation sequencing (NGS). In any of the embodiments herein, the sequencer can comprise a next generation sequencer.

In any of the embodiments herein, one or more of the plurality of sequencing reads overlap one or more gene loci within one or more subgenomic intervals in the sample. In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In any of the embodiments herein, the one or more gene loci can comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAPI, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CRCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPKI, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In any of the embodiments herein, the one or more gene loci can comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CSI, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In any of the embodiments herein, the methods disclosed herein can further comprise generating, by the one or more processors, a report indicating the determined disease score. In some embodiments, the methods disclosed herein can comprise transmitting the report to a healthcare provider. In any of the embodiments herein, the report can be transmitted via a computer network or a peer-to-peer connection.

In some aspects, disclosed herein is a method of predicting a disease state of a subject, comprising: determining, by one or more processors, a test count of methylated loci for a computationally reconstructed cfDNA fragment (CRCF) within a size range for a test sample obtained from a subject; determining, by the one or more processors, a disease score based on comparing the test count of the methylated loci to a reference count of methylated loci from one or more reference samples; and predicting, by the one or more processors, the disease state for the subject based on a comparison of the disease score to a predetermined disease score threshold.

In some embodiments, disclosed herein is a method further comprising: binning, by the one or more processors, sizes of the CRCFs; and determining, by the one or more processors, the size range based on the binned sizes. In some embodiments, disclosed herein is a method further comprising: preprocessing one or more methylation statuses of one or more nucleotides from one or more ends of sequence reads from the CRCFs. In some embodiments, the preprocessing can be based on one or more methylation statuses of other sequence reads from the CRCFs. In any of the embodiments herein, the preprocessing can comprise computationally removing the one or more nucleotides from the one or more ends of the sequence reads. In any of the embodiments herein, the preprocessing can comprise computationally correcting the one or more methylation statuses of the one or more nucleotides. In any of the embodiments herein, the disease state can indicate being at risk or having colorectal cancer (CRC). In some embodiments, the colorectal cancer can be stage 1 CRC, stage II CRC, stage III CRC, or stage IV CRC. In any of the embodiments herein, the disease state can indicate being at risk or having lung cancer. In any of the embodiments herein, the lung cancer can be stage I lung cancer, stage II lung cancer, stage III lung cancer, or stage IV lung cancer. In any of the embodiments herein, the predicted disease state can comprise a prediction of the tissue of origin for the CRCF.

In any of the embodiments herein, the disease score can be a probability from binomial testing or a Kullback-Leibler divergence. In some embodiments, the probability from the binomial testing can be based on the test count of the methylated loci and the reference count of methylated loci. In any of the embodiments herein, the disease state can be based on the probability from the binomial testing being less than the predetermined disease score threshold. In any of the embodiments herein, the predetermined disease score threshold can be 0.05. In any of the embodiments herein, the Kullback-Leibler divergence can be based on a test probability distribution of methylated loci counts and a reference probability distribution of methylated loci counts. In any of the embodiments herein, the one or more reference samples can comprise a healthy sample. In any of the embodiments herein, the test sample can be a disease sample. In any of the embodiments herein, the one or more reference samples can be obtained from a reference subject. In any of the embodiments herein, the reference count of methylated loci can be based on a synthetic reference set of sequence read count data. In some embodiments, the synthetic reference set of sequence read count data can be based on a composite profile. In some embodiments, the composite profile is based on the one or more reference samples. In any of the embodiments herein, the test sample can be obtained from the subject. In any of the embodiments herein, the test count of methylated loci can be a normalized count. In some embodiments, the normalized count can be the test count of methylated loci normalized by a count of methylated and unmethylated loci.

In any of the embodiments herein, the binning can further comprise generating an empirical distribution function of the sizes of the CRCFs. In some embodiments, the determining the size range can be based on the empirical distribution function. In any of the embodiments herein, the determining the size range can be based on Otsu thresholding. In any of the embodiments herein, the size range can correspond to a nucleosome position in a genome. In any of the embodiments herein, the size range can comprise a lower boundary of approximately 140 nucleotides in length and an upper boundary of approximately 200 nucleotides in length. In any of the embodiments herein, the size range can comprise a lower boundary of approximately 280 nucleotides in length and an upper boundary of approximately 400 nucleotides in length. In any of the embodiments herein, the size range can comprise a lower boundary of approximately 450 nucleotides in length and an upper boundary of approximately 600 nucleotides in length. In any of the embodiments herein, the size range can comprise a lower boundary of approximately 280 nucleotides in length and an upper boundary of approximately 600 nucleotides in length. In any of the embodiments herein, the determining the disease score can be used to diagnose or confirm a diagnosis of the disease state in the subject. In any of the embodiments herein, the disease state can comprise having a cancer.

In some aspects, disclosed herein is a method further comprising selecting an anti-cancer therapy to administer to the subject based on the determining of the disease score. In any of the embodiments herein, disclosed herein is a method further comprising determining an effective amount of an anti-cancer therapy to administer to the subject based on the determining of the disease score. In any of the embodiments herein, disclosed herein is a method further comprising administering an anti-cancer therapy to the subject based on the determining of the disease score. In any of the embodiments herein, the anti-cancer therapy can comprise chemotherapy, radiation therapy, immunotherapy, a targeted therapy, or surgery.

In some aspects, disclosed herein is a method for monitoring cancer progression or recurrence in a subject, the method comprising: determining a first disease score in a first test sample obtained from the subject at a first time point according to the method of any one of the embodiments herein; determining a second disease score in a second test sample obtained from the subject at a second time point; and comparing the first test sample to the second test sample, thereby monitoring the cancer progression or recurrence.

In some embodiments, the second disease score for the second test sample is determined according to the method of any of the embodiments disclosed herein. In any of the embodiments herein, disclosed herein is a method further comprising selecting an anti-cancer therapy for the subject in response to the cancer progression. In any of the embodiments herein, disclosed herein is a method further comprising administering an anti-cancer therapy to the subject in response to the cancer progression. In any of the embodiments herein, disclosed herein is a method further comprising adjusting an anti-cancer therapy for the subject in response to the cancer progression. In any of the embodiments herein, disclosed herein is a method further comprising adjusting a dosage of the anti-cancer therapy or selecting a different anti-cancer therapy in response to the cancer progression. In some embodiments, disclosed herein is a method further comprising administering the adjusted anti-cancer therapy to the subject. In any of the embodiments herein, the first time point can be before the subject has been administered an anti-cancer therapy, and wherein the second time point is after the subject has been administered the anti-cancer therapy.

In any of the embodiments herein, the subject can have a cancer, is at risk of having a cancer, is being routine tested for cancer, or is suspected of having a cancer. In any of the embodiments herein, the cancer can be a solid tumor. In any of the embodiments herein, the cancer can be a hematological cancer. In any of the embodiments herein, the anti-cancer therapy can comprise chemotherapy, radiation therapy, immunotherapy, a targeted therapy, or surgery.

In any of the embodiments herein, disclosed herein is a method further comprising determining, identifying, or applying the predicted disease score for the sample as a diagnostic value associated with the test sample. In any of the embodiments herein, disclosed herein is a method further comprising generating a genomic profile for the subject based on the determining of the predicted disease score.

In some embodiments, the genomic profile for the subject can further comprise results from a comprehensive genomic profiling (CGP) test, a gene expression profiling test, a cancer hotspot panel test, a DNA methylation test, a DNA fragmentation test, an RNA fragmentation test, or any combination thereof. In any of the embodiments herein, the genomic profile for the subject can further comprise results from a nucleic acid sequencing-based test. In any of the embodiments herein, disclosed herein is a method further comprising selecting an anti-cancer therapy, administering an anti-cancer therapy, or applying an anti-cancer therapy to the subject based on the generated genomic profile. In any of the embodiments herein, the determining of the predicted disease score for the test sample is used in making suggested treatment decisions for the subject. In any of the embodiments herein, the determining of the predicted disease score for the test sample can be used in applying or administering a treatment to the subject.

In some aspects, disclosed herein is a system comprising: one or more processors; and a memory communicatively coupled to the one or more processors and configured to store instructions that, when executed by the one or more processors, cause the system to: determine, by one or more processors, a test count of methylated loci for a computationally reconstructed cfDNA fragment (CRCF) within a size range for a test sample obtained from a subject; determine, by the one or more processors, a disease score based on comparing the determined test count of the methylated loci to a reference count of methylated loci from one or more reference samples; and predict, by the one or more processors, a disease state for the subject based on a comparison of the disease score to a predetermined disease score threshold.

In some embodiments, disclosed herein is a system comprising further instructions that, when executed by the one or more processors, cause the system to: bin sizes of the CRCFs; and determine the size range based on the binned sizes.

In any of the embodiments herein, disclosed herein is a system comprising further instructions that, when executed by the one or more processors, cause the system to: preprocess one or more methylation statuses of one or more nucleotides from one or more ends of sequence reads from the CRCFs. In some embodiments, the preprocessing can be based on one or more methylation statuses of other sequence reads from the CRCFs. In any of the embodiments herein, the preprocessing can comprise computationally removing the one or more nucleotides from the one or more ends of the sequence reads. In any of the embodiments herein, the preprocessing can comprise computationally correcting the one or more methylation statuses of the one or more nucleotides.

In some aspects, disclosed herein is a non-transitory computer-readable storage medium storing one or more programs, the one or more programs comprising instructions, which when executed by one or more processors of a system, cause the system to: determine, by one or more processors, a test count of methylated loci for a computationally reconstructed cfDNA fragment (CRCF) within a size range for a test sample obtained from the subject; determine, by the one or more processors, a disease score based on comparing the determined test count of the methylated loci to a reference count of methylated loci from one or more reference samples; and predict, by the one or more processors, a disease state for the subject based on a comparison of the disease score to a predetermined disease score threshold. In some embodiments, the non-transitory computer-readable storage medium can comprise further instructions that, when executed by the one or more processors of a system, cause the system to: bin sizes of the CRCFs; and determine the size range based on the binned sizes. In some embodiments, the non-transitory computer-readable storage medium can comprise further instructions, that, when executed by the one or more processors of a system, cause the system to: preprocess one or more methylation statuses of one or more nucleotides from one or more ends of sequence reads from the CRCFs. In some embodiments, the preprocessing can be based on one or more methylation statuses of other sequence reads from the CRCFs. In some embodiments, the preprocessing can comprise computationally removing the one or more nucleotides from the one or more ends of the sequence reads. In some embodiments, the preprocessing can comprise computationally correcting the one or more methylation statuses of the one or more nucleotides.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides an exemplary method for predicting a disease based on analyzing cfDNA fragments.

FIG. 2 provides another exemplary method for predicting a disease based on analyzing cfDNA fragments.

FIG. 3 provides another exemplary method for predicting a disease based on analyzing cfDNA fragments.

FIG. 4 depicts an exemplary computing device or system in accordance with one embodiment of the present disclosure.

FIG. 5 depicts an exemplary computer system or computer network, in accordance with some instances of the systems described herein.

FIG. 6 provides an example histogram of computationally reconstructed cfDNA fragment (CRCF) sizes.

FIG. 7A provides an example of data depicting methylation fraction as a function of cfDNA molecule size, for hypermethylated regions, from a healthy sample.

FIG. 7B provides an example of data depicting methylation fraction as a function of cfDNA molecule size, for hypermethylated regions, from a cancer sample.

FIG. 8A provides an example histogram and an example receiver-operating characteristic curve, comparing 140-200 base pair long CRCFs for healthy versus tumor samples for sequences that are hypomethylated in healthy samples, using binomial testing.

FIG. 8B provides an example histogram and an example receiver-operating characteristic curve, comparing 300-400 base pair long CRCFs molecules for healthy versus tumor samples for sequences that are hypomethylated in healthy samples, using binomial testing.

FIG. 9A provides an example histogram and an example receiver-operating characteristic curve, comparing 140-200 base pair long CRCFs for healthy versus tumor samples for sequences that are hypermethylated in healthy samples, using binomial testing.

FIG. 9B provides an example histogram an example receiver-operating characteristic curve, comparing 300-400 base pair long CRCFs for healthy versus tumor samples for sequences that are hypermethylated in healthy samples, using binomial testing.

FIG. 10 provides an example histogram of CRCF sizes.

FIG. 11A provides an example histogram and an example receiver-operating characteristic curve comparing CRCFs of all sizes for healthy versus cancer samples for sequences mapping to CpG islands, using binomial testing.

FIG. 11B provides an example histogram and an example receiver-operating characteristic curve comparing CRCFs for 140-200 base pair long cfDNA molecules for healthy versus cancer samples for sequences mapping to CpG islands, using binomial testing.

FIG. 11C provides an example histogram and an example receiver-operating characteristic curve comparing CRCFs for 200 base pair and longer CRCFs for healthy versus cancer samples for sequences mapping to CpG islands, using binomial testing.

FIG. 12A provides an example histogram and an example receiver-operating characteristic curve comparing CRCFs of all sizes for healthy versus cancer samples for sequences hypermethylated in healthy samples, using binomial testing.

FIG. 12B provides an example histogram and an example receiver-operating characteristic curve comparing CRCFs for 140-200 base pair long CRCFs for healthy versus cancer samples for sequences hypermethylated in healthy samples, using binomial testing.

FIG. 12C provides an example histogram and an example receiver-operating characteristic curve comparing CRCFs for 200 base pair and longer CRCFs for healthy versus cancer samples for sequences hypermethylated in healthy samples, using binomial testing.

FIG. 13A provides an example histogram and an example receiver-operating characteristic curve comparing CRCFs of all sizes for healthy versus cancer samples for all informative sequences, using binomial testing.

FIG. 13B provides an example histogram and an example receiver-operating characteristic curve comparing CRCFs for 140-200 base pair long CRCFs for healthy versus cancer samples for all informative sequences in healthy samples, using binomial testing.

FIG. 13C provides an example histogram and an example receiver-operating characteristic curve comparing CRCF for 200 base pair and longer CRCFs for healthy versus cancer samples for all informative sequences, using binomial testing.

FIG. 14 provides example heat-mapped tabular area under the curve (AUC) data for CRCFs of different size ranges and how predictive those different size ranges are for predicting colorectal cancer or lung cancer stages.

### DETAILED DESCRIPTION

Existing methods of predicting diseases are often limited to analyzing only a single feature of cfDNA fragments. Such a limitation can curtail the method's ability to accurately predict the disease. The methods and systems described herein integrate multiple features of cfDNA fragments, to predict a disease in a subject. The integration of multiple features allows for improved predictive accuracy for not only multiple cancer types, but for multiple stages of the cancer types, e.g., stage II lung cancer. The methods and systems described herein comprise analyzing both the methylation status, as well as the size, e.g., length, of cell-free DNA (cfDNA) molecules. More specifically, the analysis can comprise filtering out cfDNA fragment sizes that may otherwise weaken the accuracy of the prediction for the predicted disease. In performing such a filtering, the methods and systems disclosed herein improve the computational efficiency of cfDNA analysis, over some existing methods that analyze multiple cfDNA features.

The analyses can comprise computationally analyzing a size range of cfDNA molecules, and the size range can be determined based on analyzing a histogram of cfDNA molecule sizes. From the cfDNA molecules within the size range, the methylated loci can be counted. The count can be compared to other methylated loci counts, e.g., from other samples, and the comparing can include a statistical comparison. From the comparing, a disease score can be obtained, which can be used to predict the likelihood of a disease for a subject. By computationally analyzing both the methylation and the size of cfDNA fragments, the methods described herein allow for highly accurate disease predictions. It was found that by analyzing only the methylation of cfDNA fragments in a selected size range a more accurate disease state predictions can be ascertained. Given volume and complexity associated with measuring an characterizing the topography of the fragments, computerized analysis can unlock information that has been previously not realized or practically implemented.

The disclosed methods can also improve the computational efficiency of cfDNA fragment analysis. Existing methods often operate from the assumption that analyzing more data results in increased predictive power. Not all data, however, contributes equally to a method's predictive abilities. In fact, depending on the method of analyzing the data, some parts of the data can abrogate the method's predictive strength. For example, as demonstrated in the Examples herein, comparing the methylation statuses of all cfDNA molecules can, in fact, result in less accurate disease predictions, than if the methylation statuses of only cfDNA molecules of a specific size range are analyzed. Relatedly, performing a computational comparison, e.g., determining a p-value from binomial testing or a Kullback-Liebler divergence, on more data, such as all cfDNA molecules regardless of size, can be far more computationally intensive than if only a subset of the data—e.g., cfDNA molecules of a specific size range—were analyzed. Depending on the method used for the analysis, the scaling between the amount of data that is analyzed and the computational intensity of the method, e.g., the length of time or memory required to analyze the data, can be non-linear, e.g., O(n²) or O(n log n). In such cases, the ability to perform the methods described herein non-computationally is, in effect, not possible. By comparing the methylation statuses of only select data that necessarily contributes to the overall predictive power, e.g., the methylation counts of a specific size range of cfDNA molecules, the methods disclosed herein can provide non-linear improvements in computational efficiency.

The methods described herein provide a level of predictive resolution that is accurate in not only predicting whether a subject will have a disease, e.g., cancer, but is accurate in predicting whether the subject has a certain stage of the disease, e.g., cancer stage. Accordingly, the methods described herein can inform specific treatments that are tailored to the subject's disease stage or state. In doing so, the methods described herein can, for example, advise that a subject undergo more aggressive monitoring if the subject is at a higher predicted likelihood of an early-stage cancer, and in contrast, advise specific medications for subjects that are predicted to be at a later stage cancer. The predictive resolution of the methods described herein allow for the justifying of specific and tailored treatment regiments that correspond to a subject's disease progression.

Methods and systems for predicting a disease state based on analyzing cfDNA fragments are described. The method of predicting the disease state can include determining a test count of methylated loci for computationally reconstructed cfDNA fragments (CRCFs) within a size range. The test count of methylated loci can be obtained from a healthy sample or a disease sample, from a subject. The test count can be normalized and interpreted as a probability, or can be further interpreted as representing or being sampled from a statistical distribution. The test count can then be compared to a reference count of methylated loci, for example, via statistical methods, such that a Kullback-Leibler divergence, or a p-value, e.g., from a binomial test, is determined. The reference count of methylated loci can be a part of, or be based on, a subsuming dataset, such as a statistical distribution of methylation counts from a reference sample, e.g., a healthy sample from a healthy subject. The test and reference counts can be for cfDNA fragments of a specific size range, such cfDNA fragments of size 140-200 base pairs. By comparing the test and reference counts of methylated loci, a disease score that indicates a likelihood of the disease state, e.g., the likelihood that the subject has a colorectal cancer, can be outputted. That is, the disease score can predict whether the subject has or is at risk of having a disease, e.g., colorectal cancer. For example, if the disease score is greater or less than a disease score threshold, the subject can be predicted as having or being suspected of having the disease.

In some aspects, disclosed herein is a method of predicting a disease state of a subject, comprising: determining, by one or more processors, a test count of methylated loci for cfDNA fragments within a size range for a test sample obtained from a subject; determining, by the one or more processors, a disease score based on comparing the test count of the methylated loci to a reference count of methylated loci from one or more reference samples; and predicting, by the one or more processors, the disease state for the subject based on a comparison of the disease score to a predetermined disease score threshold.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

As used herein, the term “disease state” refers to a status of or relating to a disease presence or severity. The disease state need not be limited to a binary classification of having or not having the disease. The disease state can refer to a specific stage, progression, prognosis, diagnosis, risk of the disease. The disease state may be a statistical factor and/or, accordingly, a confidence, e.g., a probability, can be associated with the disease state. The confidence associated with the disease state can refer to, for example, a likelihood that the subject has the disease or will have the disease and/or at what stage that disease. The disease state and its associated confidence can be predicted, and the prediction can be based on computational techniques, e.g., machine learning models and methods. A sample, such as one or more reference samples or a test sample, can be associated with a disease state. For example, the one or more reference samples can comprise a cancer, and thus, the disease state of such one or more samples can, for example, be that of having a cancer and at what stage that cancer is likely present.

The section headings used herein are for organizational purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

Methods for Predicting a Disease State Based on Analyzing cfDNA Fragments

The disclosed methods for predicting a disease state are based on computational processes and modules configured to analyze cfDNA fragments. The analyzing leverages multiple features of the cfDNA fragments to provide a highly accurate prediction for a disease state. More specifically, the analysis integrates both the methylation statuses and the sizes, e.g., fragment lengths, of the cfDNA fragments, including computationally reconstructed cfDNA fragments (CRCFs). The CRCFs can refer to the output resulting from a merged, e.g., piecing together of sequence reads from cfDNA fragments, based on overlapping sequences of the sequence reads. In some examples, the CRCF is a result of merging together sequence reads from various regions of the fragment, e.g., paired-end sequence reads, by aligning them against a reference genome, to generate one or more merged reads. In some examples, the CRCF is a computationally generated sequence that is generated by merging together the reads associated with the same fragment during alignment, to generate a representative sequence for a nucleic acid molecule, such as a cfDNA fragment. A CRCF need not be equivalent to a literal physical cfDNA fragment molecule, but can, in some cases, represent at least a part of the sequence of a cfDNA fragment molecule. The CRCF may further be grouped together with other CRCFs to generate a consensus sequence. The disclosed methods entail analyzing the CRCFs, by performing statistical tests on the number (e.g., counts, proportions, or probabilities) of methylated or unmethylated loci, for a given CRCF. The statistical tests can be done for specifically a select size range of CRCFs. For example, a binomial test can be done to compare the proportion of methylated loci for CRCFs form a disease sample, versus CRCFs from a healthy sample, for only CRCFs that are 140-200 base pairs long. The statistical test can yield a disease score, e.g., a p-value from the statistical comparison. The disease score can then be used to determine whether a subject has a disease, based on if the disease score is greater or less than some threshold value. The methods described herein greatly increase the accuracy of disease predictions compared to methods that do not consider both the size and methylation of cfDNA fragments. Indeed, analyzing the methylated loci of only specific size ranges of cfDNA fragments vastly improves disease predictions compared to if methylated loci of all cfDNA fragment sizes are used.

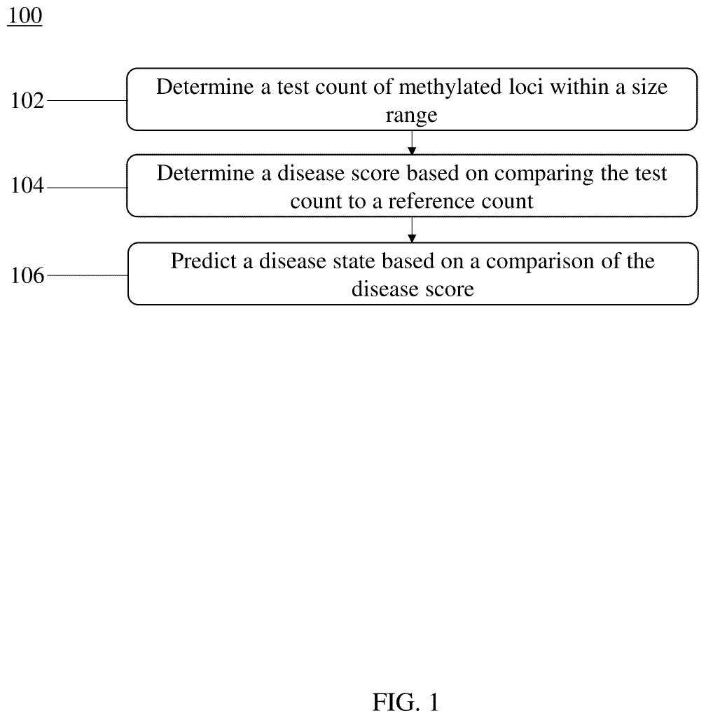

FIG. 1 shows an exemplary schematic showing general process 100 for predicting a disease state of a subject. The method of predicting a disease state can include: determining, by one or more processors, a test count of methylated loci for a computationally reconstructed cfDNA fragment (CRCF) within a size range for a test sample obtained from a subject (102); determining, by the one or more processors, a disease score based on comparing the test count of the methylated loci to a reference count of methylated loci from one or more reference samples (104); and predicting, by the one or more processors, the disease state for the subject based on a comparison of the disease score to a predetermined disease score threshold (106).

At 102 in FIG. 1, a test count of methylated loci for a computationally reconstructed cfDNA fragment (CRCF) within a size range for a test sample is obtained from the subject. The one or more reference samples can comprise a healthy sample. The test sample can be a disease sample or a sample where disease is unknown. The test sample and the reference can be obtained from the subject. That is, the one or more reference samples and the test sample can both be derived from the same subject. For example, the one or more reference samples may derive from a healthy part of the subject—that is, the one or more reference samples may be a healthy sample, e.g., a healthy blood sample. The test sample may also be derived from the subject, but from a diseased part of the subject—that is, the test sample may be a disease sample, e.g., a hepatocellular carcinoma. That is, the test sample may correspond to one or more matched internal control samples, which can be the one or more reference samples that derive from the same subject as the test sample. In addition, the test sample and the one or more reference samples that are derived from the subject as the test sample, may be collected at different timepoints from the subject, and when collected from the different timepoints, the samples may be collected from the same anatomical region of the subject. The test sample may correspond to one or more matched external control samples, which can be the one or more reference samples that derive from a common organ type from a different subject. Alternatively, the one or more reference samples and the test sample need not be matched—for example, the samples need not come from the same subject and/or come from a common organ type. For the one or more reference samples, the disease state(s) of the one or more reference samples can be predetermined. For example, the one or more reference samples can be known to be one or more healthy samples, where the disease state, e.g., the classification that the one or more samples are healthy, are, for example, ascertained by a clinician.

The test count of methylated loci can be a normalized count. The normalized count can be the test count of methylated loci normalized by a count of methylated and unmethylated loci. The test count of methylated loci can be determined for a CRCF. The test count can refer to a count of loci that are methylated for a CRCF, from a test sample. The test count of methylated loci can comprise a count of methylated nucleotides across the CRCF, a count of methylated nucleotides for one or more reads on which the CRCF is based. The methylated nucleotides can be counted, e.g., can be the test count of methylated loci. In such a case, the test count of methylated loci can be normalized by all the nucleotides for the CRCF, methylated or not, thereby determining the normalized count. For example, the normalized count can be the test count of methylated loci for the CRCF, divided by all the unmethylated and methylated loci for the CRCF. Accordingly, the normalized count can be a fraction, e.g., a value between 0 and 1, inclusive.

The size range can correspond to a nucleosome position in the genome. The size range can comprise a lower boundary of approximately 140 nucleotides in length and an upper boundary of approximately 200 nucleotides in length. In some instances, the size range of approximately 140-200 nucleotides for a cfDNA molecule can correspond to the cfDNA originating from genomic DNA wrapped around the histones of a mononucleosome. The size range can comprise a lower boundary of approximately 280 nucleotides in length and an upper boundary of approximately 400 nucleotides in length. In some instances, the size range of approximately 280-400 nucleotides for a cfDNA molecule can correspond to the cfDNA originating from genomic DNA wrapped around the histones of a dinucleosome. The size range can comprise a lower boundary of approximately 450 nucleotides in length and an upper boundary of approximately 600 nucleotides in length. In some instances, the size range of approximately 450-600 nucleotides can correspond to the cfDNA originating from genomic DNA wrapped around the histones of a trinucleosome. The size range can comprise a lower boundary of approximately 280 nucleotides in length and an upper boundary of approximately 600 nucleotides in length. In some instances, the size range of approximately 450-600 nucleotides can correspond to the cfDNA originating from genomic DNA wrapped around the histones of a polynucleosome, e.g., a dinucleosome or a trinucleosome. The size range can be predetermined, regardless of a distribution of cfDNA fragment sizes. Alternatively, the size range can be determined based on a distribution of cfDNA fragment sizes.

At 104 in FIG. 1, a disease score is determined based on comparing the determined test count of the methylated loci against a reference count of methylated loci from a test sample. The disease score can be a probability from, for example, a binomial testing or a Kullback-Leibler divergence. The probability from the binomial testing can be based on the determined test count of the methylated loci and the reference count of methylated loci. The probability from the binomial testing can be determined by an expression, such as:

$$P ( x ) = {\sum }_{i = x}^{n} \frac{n !}{( n - x ) ! x !} {p}^{x} {q}^{n - x}$$

wherein, i is an index of summation; x is the amount of methylation for an observed CRCF from the test sample, e.g., disease sample, and in general, can be the reference count of methylated loci for the CRCF; P(x) is the probability that the amount of methylation seen in an observed CRCF is significantly different from a corresponding reference sequence, e.g., a reference CRCF sequence, from one or more reference samples; x is the total number of methylated loci for the observed CRCF; n is the total number of loci, methylated or not, for the observed CRCF; p is the probability of observing the amount of methylation in the corresponding reference sequence, e.g., the reference CRCF sequence; p can be understood as the normalized reference count of methylated loci for the reference CRCF, where the count of methylated loci in the reference CRCF is divided by the count of total loci in the reference CRCF; q is 1−p, i.e., q is the complementary probability of p. The probability from the binomial testing can be understood as the disease score.

The Kullback-Leibler divergence can be based on a test probability distribution of methylated loci counts and a reference probability distribution of methylated loci counts. The Kullback-Leibler divergence can be understood, in the information theory sense, as the relative entropy between a test probability distribution, and a reference probability distribution. That is, the Kullback-Leibler divergence can be understood as the average difference in the number of bits necessary for encoding samples of the test probability distribution when using a code optimized for the reference probability distribution, rather than a code optimized for the test probability distribution. Thus, the Kullback-Leibler divergence is asymmetric. In some general sense, the Kullback-Leibler divergence can be understood as how different the test probability distribution is from the reference probability distribution. For discrete probability distributions, the Kullback-Leibler divergence can be determined by an expression, such as:

$${D}_{KL} ( P ‖ Q ) = {\sum }_{x ϵ X} P ( x ) log ( \frac{P ( x )}{Q ( x )} )$$

wherein X is the sample space, P(x) is test probability distribution—that is, the probability distribution of the amount of methylation for one or more observed computationally reconstructed cfDNA fragments (CRCFs) from the test sample, e.g., disease sample, and in general, P(x) can be the probability distribution based on a test count of methylated loci; [symbol] (x) is the reference probability distribution, e.g., the probability distribution of the amount of methylation for one or more CRCFs from the one or more reference samples, e.g., one or more healthy samples, and in general, [symbol] (x) can be the probability distribution based on a reference count of methylated loci; and D_{KL}(P|Q) is the Kullback-Leibler divergence of the test probability distribution P from the reference probability distribution [symbol] .

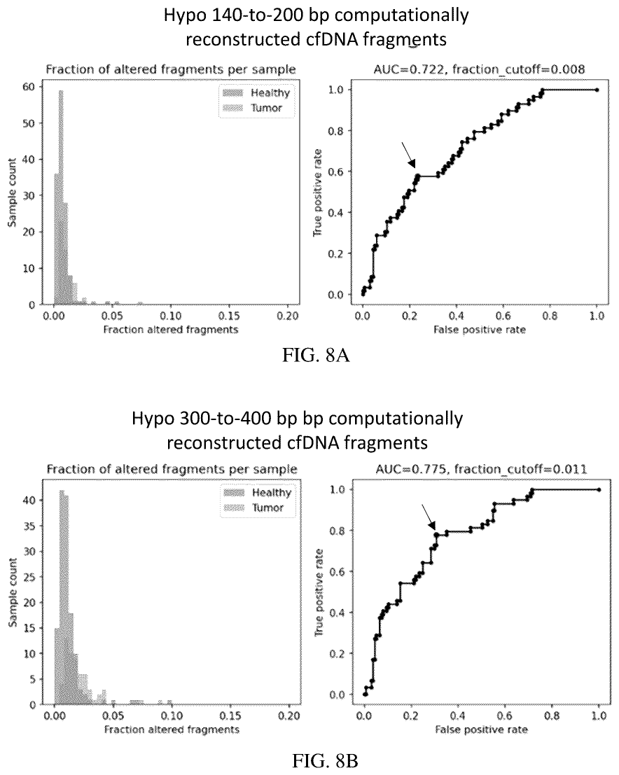

Once a disease score is calculated for a CRCF, e.g., a p-value from a binomial test, or a Kullback-Leibler divergence, the disease score can be compared to a predetermined disease score threshold. If the disease score is greater than or less than the predetermined disease score threshold, the CRCF can be assigned a flag that designates the CRCF as being significantly different, i.e., significantly altered, from the one or more reference samples, e.g., reference probability distribution. For a test sample, the fraction of fragments that are significantly different, i.e., significantly altered, from the one or more reference samples can be determined, and optionally, visualized. FIGS. 8A-9B, 11A-13C, provide example visualizations, in the form of histograms, of the fraction of fragments that are significantly altered from the one or more reference samples.

At 106 in FIG. 1, a disease state for a subject is predicted based on a comparison of the disease score to a predetermined disease score threshold. The disease can be based on the probability from the binomial testing being less than the predetermined disease score threshold. The predetermined disease score threshold can be 0.05, and the predetermined disease score threshold can be a positive value. For example, from the binomial testing, P(x) can be less than 0.05, which can be understood to mean that, based on the observed count of methylated loci from the healthy sample, the probability of seeing the observed count of methylated loci from the disease sample is less than 0.05, which can be understood to be unlikely. Therefore, the disease score can be understood to mean that the observed count of methylated loci from the disease sample is unlikely to resemble the methylation patterns of the healthy sample, which may indicate that the methylation pattern from the disease sample is indeed indicative of a disease. In some instances, the disease can be colorectal cancer (CRC). The colorectal cancer can be stage I CRC, stage II CRC, stage III CRC, or stage IV CRC. The disease can be lung cancer. The lung cancer can be stage I lung cancer, stage II lung cancer, stage III lung cancer, or stage IV lung cancer. The predicting the disease can be at a resolution, such that accurate predictions are provided for specific stages of cancer, such as for stage I CRC or stage III lung cancer. Given that the predicting the disease can include the accurate predicting of cancer stages, cancer stage-specific treatments can be provided, based on the accurate cancer stage-specific predictions. For example, high disease score-based predictions for early-stage cancers can inform first line treatments or resections, or more aggressive monitoring of the patient. In some examples, the predicted disease state comprises a prediction of the tissue of origin for the CRCF. That is, analyzing the CRCF can be used to predict the tissue from which the cfDNA fragment corresponding to the CRCF, originated. For example, based on features of the CRCF, it could be predicted that the cfDNA fragment may have originated, e.g., broken off, from genomic DNA of a liver cell.

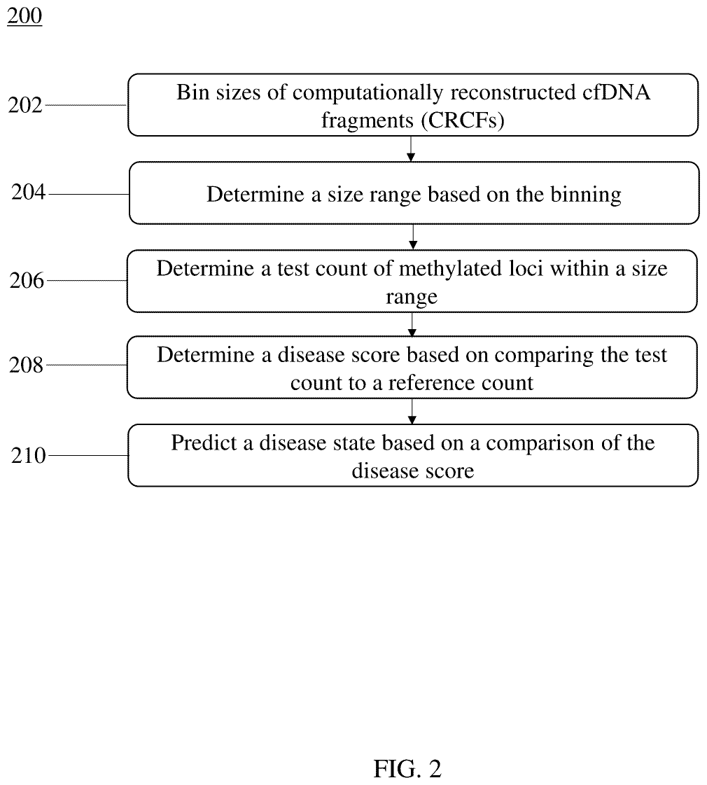

FIG. 2 shows another exemplary method 200 for predicting a disease state of a subject. The method of predicting a disease state can include: binning by one or more processors, sizes of the cfDNA fragments (202); determining, by the one or more processors, the size range based on the binned sizes (204); determining, by the one or more processors, a test count of methylated loci for cfDNA fragments within a size range for a test sample obtained from a subject (206); determining, by the one or more processors, a disease score based on comparing the test count of the methylated loci to a reference count of methylated loci from one or more reference samples (208); and predicting, by the one or more processors, the disease state for the subject based on a comparison of the disease score to a predetermined disease score threshold (210).

At 202 in FIG. 2, sizes of the cfDNA fragments are binned. The binning can further comprise generating an empirical distribution function of the sizes of the cfDNA fragments. The generating the empirical distribution function need not be confined to a visual representation, e.g., a histogram plot or an empirical cumulative distribution function curve. The empirical distribution function can be understood as comprising data, e.g., the counts of methylated loci, which can be assumed as being derived from a sampling from an underlying statistical distribution. The binning of the counts of methylated loci, e.g., the test count of methylated loci, can be understood as the sorting of the methylated loci counts according to the sizes of the computationally reconstructed cfDNA fragments (CRCFs), such that each bin comprises the one or more counts of methylated loci of one or more CRCFs. The bins can be contiguous, such that the exclusive upper boundary of one bin is the same value as the inclusive lower boundary of an adjacent bin. A visual representation of the binning can comprise a histogram, where each discrete bar of the histogram represents a bin of CRCF sizes. Thus, the collection of bins can form the histogram of CRCF sizes.

At 204 in FIG. 2, the size range is determined based on the binned sizes. That is, from the binned sizes, a subset of bins, e.g., one or more bins, can be selected. The subset of bins can comprise several bins for which, when the bins are sorted, the upper boundary of the preceding bin is adjacent to the lower boundary of the proceeding bin. The determining the size range can be based on a shape of the empirical distribution function. For example, the determining the size range based on the many bins of size ranges can be visually represented as a histogram, and a subset of the histogram is selected. Determining the size range based on the shape of the empirical distribution function can include, for example, features of the empirical distribution function such as the number and broadness of the peaks of the distribution, the skewedness of the distribution, the uniformity of the distribution, and/or the symmetry of the distribution. The determining the size range can be based on Otsu thresholding. For example, in terms of a visual representation, the subset of the histogram of cfDNA fragment sizes can be determined by having the boundaries of the histogram subset be based on Otsu thresholding. Otsu thresholding is an automated method of thresholding that relies on a brute force search for a threshold that minimizes the intra-class variance or maximizes the inter-class variance, where class refers to the parts of the distribution above and below the Otsu threshold. For example, the classes can refer to counts of methylated loci above and below the Otsu threshold. The Otus threshold can, for example, be expressed as:

$${σ}_{ω}^{2} ( t ) = {ω}_{0} ( t ) {σ}_{0}^{2} ( t ) + {ω}_{1} ( t ) {σ}_{1}^{2} ( t )$$

wherein ω₀ is a weight that describes the probability of the class 0 being separated by the threshold, t; ω₁ is a weight that describes the probability of class 1 being separated by the threshold, t;

$${σ}_{0}^{2}$$

is the variance of class 0; and

$${σ}_{1}^{2}$$

is the variance of class 1. The weights as a function of the threshold can be determined from a discretized probability distribution, such as a histogram of L bins, according to expressions such as:

$${ω}_{0} ( t ) = {\sum }_{i = 0}^{t - 1} p ( i )$$

$$and$$

$${ω}_{1} ( t ) = {\sum }_{i = t}^{L - 1} p ( i )$$

wherein i is a class, e.g., methylated loci count for a bin, and p(i) refers to the probability of the class.

At 206 in FIG. 2, a test count of methylated loci for a computationally reconstructed cfDNA fragment (CRCF) within a size range for a test sample is obtained from the subject. The one or more reference samples can comprise a healthy sample. The test sample can be a disease sample or a sample where disease is unknown. The test sample and the reference can be obtained from the subject. That is, the one or more reference samples and the test sample can both be derived from the same subject. For example, the one or more reference samples may derive from a healthy part of the subject—that is, the one or more reference samples may be a healthy sample, e.g., a healthy blood sample. The test sample may also be derived from the subject, but from a diseased part of the subject—that is, the test sample may be a disease sample, e.g., a hepatocellular carcinoma. That is, the test sample may correspond to one or more matched internal control samples, which can be the one or more reference samples that derive from the same subject as the test sample. In addition, the test sample and the one or more reference samples that are derived from the subject as the test sample, may be collected at different timepoints from the subject, and when collected from the different timepoints, the samples may be collected from the same anatomical region of the subject. The test sample may correspond to one or more matched external control samples, which can be the one or more reference samples that derive from a common organ type from a different subject. Alternatively, the one or more reference samples and the test sample need not be matched—for example, the samples need not come from the same subject and/or come from a common organ type. For the one or more reference samples, the disease state(s) of the one or more reference samples can be predetermined. For example, the one or more reference samples can be known to be one or more healthy samples, where the disease state, e.g., the classification that the one or more samples are healthy, are, for example, ascertained by a clinician.

The test count of methylated loci can be a normalized count. The normalized count can be the test count of methylated loci normalized by a count of methylated and unmethylated loci. The test count of methylated loci can be determined for a CRCF. The test count can refer to a count of loci that are methylated for a CRCF, from a test sample. The test count of methylated loci can comprise a count of methylated nucleotides across the CRCF, a count of methylated nucleotides for one or more reads on which the CRCF is based. The methylated nucleotides can be counted, e.g., can be the test count of methylated loci. In such a case, the test count of methylated loci can be normalized by all the nucleotides for the CRCF, methylated or not, thereby determining the normalized count. For example, the normalized count can be the test count of methylated loci for the CRCF, divided by all the unmethylated and methylated loci for the CRCF. Accordingly, the normalized count can be a fraction, e.g., a value between 0 and 1, inclusive.

The size range can correspond to a nucleosome position in the genome. The size range can comprise a lower boundary of approximately 140 nucleotides in length and an upper boundary of approximately 200 nucleotides in length. In some instances, the size range of approximately 140-200 nucleotides for a cfDNA molecule can correspond to the cfDNA originating from genomic DNA wrapped around the histones of a mononucleosome. The size range can comprise a lower boundary of approximately 280 nucleotides in length and an upper boundary of approximately 400 nucleotides in length. In some instances, the size range of approximately 280-400 nucleotides for a cfDNA molecule can correspond to the cfDNA originating from genomic DNA wrapped around the histones of a dinucleosome. The size range can comprise a lower boundary of approximately 450 nucleotides in length and an upper boundary of approximately 600 nucleotides in length. In some instances, the size range of approximately 450-600 nucleotides can correspond to the cfDNA originating from genomic DNA wrapped around the histones of a trinucleosome. The size range can comprise a lower boundary of approximately 280 nucleotides in length and an upper boundary of approximately 600 nucleotides in length. In some instances, the size range of approximately 450-600 nucleotides can correspond to the cfDNA originating from genomic DNA wrapped around the histones of a polynucleosome, e.g., a dinucleosome or a trinucleosome. The size range can be predetermined, regardless of a distribution of cfDNA fragment sizes. Alternatively, the size range can be determined based on a distribution of cfDNA fragment sizes.

At 208 in FIG. 2, a disease score is determined based on comparing the determined test count of the methylated loci against a reference count of methylated loci from a test sample. The disease score can be a probability from binomial testing or a Kullback-Leibler divergence. The probability from the binomial testing can be based on the determined test count of the methylated loci and the reference count of methylated loci. The probability from the binomial testing can be determined by an expression, such as:

$$P ( x ) = {\sum }_{i = x}^{n} \frac{n !}{( n - x ) ! x !} {p}^{x} {q}^{n - x}$$

wherein, i is an index of summation; x is the amount of methylation for observed cfDNA fragments from the test sample, e.g., disease sample, that map against a genomic region of interest, and in general, can be the reference count of methylated loci; P(x) is the probability of observing at least the amount of methylation seen from the observed cfDNA fragments, and in general, can be the normalized test count of methylated loci; n is the total number of observed cfDNA fragments from the test sample that map against the genomic region of interest, and in some instantiations, can describe the reference count of methylated loci; p is the probability of observing the amount of methylation according to the first sample, e.g., the normalized test count of methylated loci, where the test count of methylated loci can be normalized, e.g., divided, by the total count of methylated loci; q is 1−p, i.e., q is the complementary probability of p. The probability from the binomial testing can be understood as the probability of observing the reference count of methylated loci, e.g., the probability of observing the methylated loci count from a disease sample, based on the test count of methylated loci, e.g., based on the methylated loci count from a healthy sample. The probability from the binomial testing can be understood as the disease score.

The reference count of methylated loci for the CRCF need not be limited to CRCFs of a size range, such as the size range used to select for the test count of methylated loci for the test sample. That is, the reference count of methylated loci for the CRCF can, for example, be determined based on any of the CRCFs available, regardless of CRCF size. The reference count of methylated loci can be based on a synthetic reference set of sequence read count data. The synthetic reference set of sequence read count data can be based on a composite profile. The composite profile can be based on the one or more reference samples. In some instances, the synthetic reference set of sequence read count data can be based on one or more methods where the synthetic reference set of sequence read count data is generated in accordance with publication PCT/US2023/069150. The one or more methods for generating the synthetic reference set of sequence read count data can be referred to as a method for generating a “panel of normal” sequence read count data. This “panel of normal” method of generating the synthetic reference set of sequence read count data can comprise performing a multivariate analysis, e.g., principal component analysis (PCA) on the sequence read count data from one or more reference samples, to capture and characterize variation across the one or more reference samples. From the multivariate analysis, the components that explain the most variation can be used to filter out noise from the one or more reference samples. For example, the elements of the data vectors projected onto the top principal component vectors can be filtered for, in the case that the sequence read count data is subject to a dimensionality reduction technique, such as PCA. The resulting noise-filtered data can be used to reconstruct a composite profile, which can be understood to be based on the analyzed synthetic reference set of sequence read count data.

The Kullback-Leibler divergence can be based on a test probability distribution of methylated loci counts and a reference probability distribution of methylated loci counts. The Kullback-Leibler divergence can be understood, in the information theory sense, as the relative entropy between a test probability distribution, and a reference probability distribution. That is, the Kullback-Leibler divergence can be understood as the average difference in the number of bits necessary for encoding samples of the test probability distribution when using a code optimized for the reference probability distribution, rather than a code optimized for the test probability distribution. Thus, the Kullback-Leibler divergence is asymmetric. In some general sense, the Kullback-Leibler divergence can be understood as how different the test probability distribution is from the reference probability distribution. For discrete probability distributions, the Kullback-Leibler divergence can be determined by an expression, such as:

$${D}_{KL} ( P ‖ Q ) = {\sum }_{x ϵ X} P ( x ) log ( \frac{P ( x )}{Q ( x )} )$$

wherein X is the sample space, P(x) is test probability distribution—that is, the probability distribution of the amount of methylation for one or more observed computationally reconstructed cfDNA fragments (CRCFs) from the test sample, e.g., disease sample, and in general, P(x) can be the probability distribution based on a test count of methylated loci; [symbol] (x) is the reference probability distribution, e.g., the probability distribution of the amount of methylation for one or more CRCFs from the one or more reference samples, e.g., one or more healthy samples, and in general, [symbol] (x) can be the probability distribution based on a reference count of methylated loci; and D_{KL}(P∥Q) is the Kullback-Leibler divergence of the test probability distribution P from the reference probability distribution [symbol] .

Once a disease score is calculated for a CRCF, e.g., a p-value from a binomial test, or a Kullback-Leibler divergence, the disease score can be compared to a predetermined disease score threshold. If the disease score is greater than or less than the predetermined disease score threshold, the CRCF can be assigned a flag that designates the CRCF as being significantly different, i.e., significantly altered, from the one or more reference samples, e.g., reference probability distribution. For a test sample, the fraction of fragments that are significantly different, i.e., significantly altered, from the one or more reference samples can be determined, and optionally, visualized. FIGS. 8A-9B, 11A-13C, provide example visualizations, in the form of histograms, of the fraction of fragments that are significantly altered from the one or more reference samples.

At 210 in FIG. 2, a disease state for a subject is predicted based on a comparison of the disease score to a predetermined disease score threshold. The disease can be based on the probability from the binomial testing being less than the predetermined disease score threshold. The predetermined disease score threshold can be for example 0.05, and the predetermined disease score threshold can be a positive value or a negative value. For example, from the binomial testing, P(x) can be less than 0.05, which can be understood to mean that, based on the observed count of methylated loci from the healthy sample, the probability of seeing the observed count of methylated loci from the disease sample is less than 0.05, which can be understood to be unlikely. Therefore, the disease score can be understood to mean that the observed count of methylated loci from the disease sample is unlikely to resemble the methylation patterns of the healthy sample, which may indicate that the methylation pattern from the disease sample is indeed indicative of a disease. In some instances, the disease can be colorectal cancer (CRC). The colorectal cancer can be stage I CRC, stage II CRC, stage III CRC, or stage IV CRC. The disease can be lung cancer. The lung cancer can be stage I lung cancer, stage II lung cancer, stage III lung cancer, or stage IV lung cancer. The predicting the disease can be at a resolution, such that accurate predictions are provided for specific stages of cancer, such as for stage I CRC or stage III lung cancer. Given that the predicting the disease can include the accurate predicting of cancer stages, cancer stage-specific treatments can be provided, based on the accurate cancer stage-specific predictions. For example, high disease score-based predictions for early-stage cancers can inform first line treatments or resections, or more aggressive monitoring of the patient. In some examples, the predicted disease state comprises a prediction of the tissue of origin for the CRCF. That is, analyzing the CRCF can be used to predict the tissue from which the cfDNA fragment corresponding to the CRCF, originated. For example, based on features of the CRCF, it could be predicted that the cfDNA fragment may have originated, e.g., broken off, from genomic DNA of a liver cell.

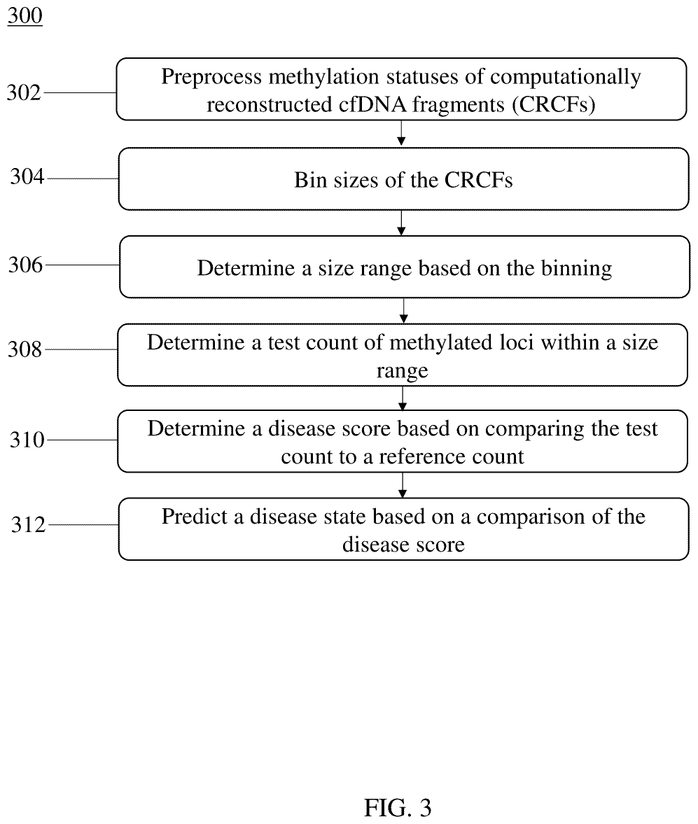

FIG. 3 shows an exemplary schematic showing general process 300 for predicting a disease state of a subject. The method of predicting a disease can include: correcting one or more methylation statuses of one or more ends of sequence reads from the cfDNA fragments, based on one or more methylation statuses of other sequence reads from the cfDNA fragments (302); binning by one or more processors, sizes of the cfDNA fragments (304); determining, by the one or more processors, the size range based on the binned sizes (306); determining, by the one or more processors, a test count of methylated loci for cfDNA fragments within a size range for a test sample obtained from a subject (308); determining, by the one or more processors, a disease score based on comparing the test count of the methylated loci to a reference count of methylated loci from one or more reference samples (310); and predicting, by the one or more processors, the disease state for the subject based on a comparison of the disease score to a predetermined disease score threshold (312).

At 302 in FIG. 3, one or more methylation statuses of one or more nucleotides from one or more ends of sequence reads from the CRCFs are preprocessed. The preprocessing can be based on one or more methylation statuses of other sequence reads from the CRCFs. The preprocessing can comprise computationally removing the one or more nucleotides from the one or more ends of the sequence reads. The preprocessing can comprise computationally correcting the one or more methylation statuses of the one or more nucleotides. In some instances, the ends of sequence reads can be subject to a systematic positive bias for unmethylated bases on the ends of the sequence reads. The systematic positive bias can happen because some cfDNA fragments, such as cfDNA molecules, possess jagged ends, which, by default, are filled in during the sequencing library processing steps via double-stranded polymerases and non-methylated nucleotides. As a result, the blunting of jagged-end cfDNA molecules can positively bias non-methylation of cfDNA sequencing ends. Computational preprocessing methods can be used to address, in silico, the positive bias of unmethylated loci on the ends of sequence reads. For example, a contiguous sequence of unmethylated loci on the ends of sequence reads can indicate that at least some number of the unmethylated loci from the contiguous sequence should be removed, or corrected to methylated loci. Examples of methods for detecting methylation biases in silico are described with respect to PCT International Application No. PCT/US2024/015952 and/or U.S. Patent Application No. 63/608,743.

At 304 in FIG. 3, sizes of the cfDNA fragments are binned. The binning can further comprise generating an empirical distribution function of the sizes of the cfDNA fragments. The generating the empirical distribution function need not be confined to a visual representation, e.g., a histogram plot or an empirical cumulative distribution function curve. The empirical distribution function can be understood as comprising data, e.g., the counts of methylated loci, which can be assumed as being derived from a sampling from an underlying statistical distribution. The binning of the counts of methylated loci, e.g., the test count of methylated loci, can be understood as the sorting of the methylated loci counts according to the sizes of the computationally reconstructed cfDNA fragments (CRCFs), such that each bin comprises the one or more counts of methylated loci of one or more CRCFs. The bins can be contiguous, such that the exclusive upper boundary of one bin is the same value as the inclusive lower boundary of an adjacent bin. A visual representation of the binning can comprise a histogram, where each discrete bar of the histogram represents a bin of CRCF sizes. Thus, the collection of bins can form the histogram of CRCF sizes.

At 306 in FIG. 3, the size range is determined based on the binned sizes. That is, from the binned sizes, a subset of bins, e.g., one or more bins, can be selected. The subset of bins can comprise several bins for which, when the bins are sorted, the upper boundary of the preceding bin is adjacent to the lower boundary of the proceeding bin. The determining the size range can be based on a shape of the empirical distribution function. For example, the determining the size range based on the many bins of size ranges can be visually represented as a histogram, and a subset of the histogram is selected. Determining the size range based on the shape of the empirical distribution function can include, for example, features of the empirical distribution function such as the number and broadness of the peaks of the distribution, the skewedness of the distribution, the uniformity of the distribution, and/or the symmetry of the distribution. The determining the size range can be based on Otsu thresholding. For example, in terms of a visual representation, the subset of the histogram of cfDNA fragment sizes can be determined by having the boundaries of the histogram subset be based on Otsu thresholding. Otsu thresholding is an automated method of thresholding that relies on a brute force search for a threshold that minimizes the intra-class variance or maximizes the inter-class variance, where class refers to the parts of the distribution above and below the Otsu threshold. For example, the classes can refer to counts of methylated loci above and below the Otsu threshold. The Otus threshold can, for example, be expressed as:

$${σ}_{ω}^{2} ( t ) = {ω}_{0} ( t ) {σ}_{0}^{2} ( t ) + {ω}_{1} ( t ) {σ}_{1}^{2} ( t )$$

wherein ω₀ is a weight that describes the probability of the class 0 being separated by the threshold, t; ω₁ is a weight that describes the probability of class 1 being separated by the threshold, t;

$${σ}_{0}^{2}$$

is the variance of class 0; and

$${σ}_{1}^{2}$$

is the variance of class 1. The weights as a function of the threshold can be determined from a discretized probability distribution, such as a histogram of L bins, according to expressions such as:

$${ω}_{0} ( t ) = {\sum }_{i = 0}^{t - 1} p ( i )$$

$$and$$

$${ω}_{1} ( t ) = {\sum }_{i = t}^{L - 1} p ( i )$$

wherein i is a class, e.g., methylated loci count for a bin, and p(i) refers to the probability of the class.

At 308 in FIG. 3, a test count of methylated loci for a computationally reconstructed cfDNA fragment (CRCF) within a size range for a test sample is obtained from the subject. The one or more reference samples can comprise a healthy sample. The test sample can be a disease sample or a sample where disease is unknown. The test sample and the reference can be obtained from the subject. That is, the one or more reference samples and the test sample can both be derived from the same subject. For example, the one or more reference samples may derive from a healthy part of the subject—that is, the one or more reference samples may be a healthy sample, e.g., a healthy blood sample. The test sample may also be derived from the subject, but from a diseased part of the subject—that is, the test sample may be a disease sample, e.g., a hepatocellular carcinoma. That is, the test sample may correspond to one or more matched internal control samples, which can be the one or more reference samples that derive from the same subject as the test sample. In addition, the test sample and the one or more reference samples that are derived from the subject as the test sample, may be collected at different timepoints from the subject, and when collected from the different timepoints, the samples may be collected from the same anatomical region of the subject. The test sample may correspond to one or more matched external control samples, which can be the one or more reference samples that derive from a common organ type from a different subject. Alternatively, the one or more reference samples and the test sample need not be matched—for example, the samples need not come from the same subject and/or come from a common organ type. For the one or more reference samples, the disease state(s) of the one or more reference samples can be predetermined. For example, the one or more reference samples can be known to be one or more healthy samples, where the disease state, e.g., the classification that the one or more samples are healthy, are, for example, ascertained by a clinician.

The test count of methylated loci can be a normalized count. The normalized count can be the test count of methylated loci normalized by a count of methylated and unmethylated loci. The test count of methylated loci can be determined for a CRCF. The test count can refer to a count of loci that are methylated for a CRCF, from a test sample. The test count of methylated loci can comprise a count of methylated nucleotides across the CRCF, a count of methylated nucleotides for one or more reads on which the CRCF is based. The methylated nucleotides can be counted, e.g., can be the test count of methylated loci. In such a case, the test count of methylated loci can be normalized by all the nucleotides for the CRCF, methylated or not, thereby determining the normalized count. For example, the normalized count can be the test count of methylated loci for the CRCF, divided by all the unmethylated and methylated loci for the CRCF. Accordingly, the normalized count can be a fraction, e.g., a value between 0 and 1, inclusive.

The size range can correspond to a nucleosome position in the genome. The size range can comprise a lower boundary of approximately 140 nucleotides in length and an upper boundary of approximately 200 nucleotides in length. In some instances, the size range of approximately 140-200 nucleotides for a cfDNA molecule can correspond to the cfDNA originating from genomic DNA wrapped around the histones of a mononucleosome. The size range can comprise a lower boundary of approximately 280 nucleotides in length and an upper boundary of approximately 400 nucleotides in length. In some instances, the size range of approximately 280-400 nucleotides for a cfDNA molecule can correspond to the cfDNA originating from genomic DNA wrapped around the histones of a dinucleosome. The size range can comprise a lower boundary of approximately 450 nucleotides in length and an upper boundary of approximately 600 nucleotides in length. In some instances, the size range of approximately 450-600 nucleotides can correspond to the cfDNA originating from genomic DNA wrapped around the histones of a trinucleosome. The size range can comprise a lower boundary of approximately 280 nucleotides in length and an upper boundary of approximately 600 nucleotides in length. In some instances, the size range of approximately 450-600 nucleotides can correspond to the cfDNA originating from genomic DNA wrapped around the histones of a polynucleosome, e.g., a dinucleosome or a trinucleosome. The size range can be predetermined, regardless of a distribution of cfDNA fragment sizes. Alternatively, the size range can be determined based on a distribution of cfDNA fragment sizes.

At 208 in FIG. 2, a disease score is determined based on comparing the determined test count of the methylated loci against a reference count of methylated loci from a test sample. The disease score can be a probability from binomial testing or a Kullback-Leibler divergence. The probability from the binomial testing can be based on the determined test count of the methylated loci and the reference count of methylated loci. The probability from the binomial testing can be determined by an expression, such as:

$$P ( x ) = {\sum }_{i = x}^{n} \frac{n !}{( n - x ) ! x !} {p}^{x} {q}^{n - x}$$

wherein, i is an index of summation; x is the amount of methylation for observed cfDNA fragments from the test sample, e.g., disease sample, that map against a genomic region of interest, and in general, can be the reference count of methylated loci; P(x) is the probability of observing at least the amount of methylation seen from the observed cfDNA fragments, and in general, can be the normalized test count of methylated loci; n is the total number of observed cfDNA fragments from the test sample that map against the genomic region of interest, and in some instantiations, can describe the reference count of methylated loci; p is the probability of observing the amount of methylation according to the first sample, e.g., the normalized test count of methylated loci, where the test count of methylated loci can be normalized, e.g., divided, by the total count of methylated loci; q is 1−p, i.e., q is the complementary probability of p. The probability from the binomial testing can be understood as the probability of observing the reference count of methylated loci, e.g., the probability of observing the methylated loci count from a disease sample, based on the test count of methylated loci, e.g., based on the methylated loci count from a healthy sample. The probability from the binomial testing can be understood as the disease score.

The reference count of methylated loci for the CRCF need not be limited to CRCFs of a size range, such as the size range used to select for the test count of methylated loci for the test sample. That is, the reference count of methylated loci for the CRCF can, for example, be determined based on any of the CRCFs available, regardless of CRCF size. The reference count of methylated loci can be based on a synthetic reference set of sequence read count data. The synthetic reference set of sequence read count data can be based on a composite profile. The composite profile can be based on the one or more reference samples. In some instances, the synthetic reference set of sequence read count data can be based on one or more methods where the synthetic reference set of sequence read count data is generated in accordance with publication PCT/US2023/069150. The one or more methods for generating the synthetic reference set of sequence read count data can be referred to as a method for generating a “panel of normal” sequence read count data. This “panel of normal” method of generating the synthetic reference set of sequence read count data can comprise performing a multivariate analysis, e.g., principal component analysis (PCA) on the sequence read count data from one or more reference samples, to capture and characterize variation across the one or more reference samples. From the multivariate analysis, the components that explain the most variation can be used to filter out noise from the one or more reference samples. For example, the elements of the data vectors projected onto the top principal component vectors can be filtered for, in the case that the sequence read count data is subject to a dimensionality reduction technique, such as PCA. The resulting noise-filtered data can be used to reconstruct a composite profile, which can be understood to be based on the analyzed synthetic reference set of sequence read count data.

The Kullback-Leibler divergence can be based on a test probability distribution of methylated loci counts and a reference probability distribution of methylated loci counts. The Kullback-Leibler divergence can be understood, in the information theory sense, as the relative entropy between a test probability distribution, and a reference probability distribution. That is, the Kullback-Leibler divergence can be understood as the average difference in the number of bits necessary for encoding samples of the test probability distribution when using a code optimized for the reference probability distribution, rather than a code optimized for the test probability distribution. Thus, the Kullback-Leibler divergence is asymmetric. In some general sense, the Kullback-Leibler divergence can be understood as how different the test probability distribution is from the reference probability distribution. For discrete probability distributions, the Kullback-Leibler divergence can be determined by an expression, such as:

$${D}_{KL} ( P ‖ Q ) = {\sum }_{x ϵ X} P ( x ) log ( \frac{P ( x )}{Q ( x )} )$$

wherein X is the sample space, P(x) is test probability distribution—that is, the probability distribution of the amount of methylation for one or more observed computationally reconstructed cfDNA fragments (CRCFs) from the test sample, e.g., disease sample, and in general, P(x) can be the probability distribution based on a test count of methylated loci; [symbol] (x) is the reference probability distribution, e.g., the probability distribution of the amount of methylation for one or more CRCFs from the one or more reference samples, e.g., one or more healthy samples, and in general, [symbol] (x) can be the probability distribution based on a reference count of methylated loci; and D_{KL}(P∥Q) is the Kullback-Leibler divergence of the test probability distribution P from the reference probability distribution [symbol] .

Once a disease score is calculated for a CRCF, e.g., a p-value from a binomial test, or a Kullback-Leibler divergence, the disease score can be compared to a predetermined disease score threshold. If the disease score is greater than or less than the predetermined disease score threshold, the CRCF can be assigned a flag that designates the CRCF as being significantly different, i.e., significantly altered, from the one or more reference samples, e.g., reference probability distribution. For a test sample, the fraction of fragments that are significantly different, i.e., significantly altered, from the one or more reference samples can be determined, and optionally, visualized. FIGS. 8A-9B, 11A-13C, provide example visualizations, in the form of histograms, of the fraction of fragments that are significantly altered from the one or more reference samples.

At 312 in FIG. 3, a disease state for a subject is predicted based on a comparison of the disease score to a predetermined disease score threshold. The disease can be based on the probability from the binomial testing being less than the predetermined disease score threshold. The predetermined disease score threshold can be 0.05, and the predetermined disease score threshold can be a positive value or a negative value. For example, from the binomial testing, P(x) can be less than 0.05, which can be understood to mean that, based on the observed count of methylated loci from the healthy sample, the probability of seeing the observed count of methylated loci from the disease sample is less than 0.05, which can be understood to be unlikely. Therefore, the disease score can be understood to mean that the observed count of methylated loci from the disease sample is unlikely to resemble the methylation patterns of the healthy sample, which may indicate that the methylation pattern from the disease sample is indeed indicative of a disease. In some instances, the disease can be colorectal cancer (CRC). The colorectal cancer can be stage I CRC, stage II CRC, stage III CRC, or stage IV CRC. The disease can be lung cancer. The lung cancer can be stage I lung cancer, stage II lung cancer, stage III lung cancer, or stage IV lung cancer. The predicting the disease can be at a resolution, such that accurate predictions are provided for specific stages of cancer, such as for stage I CRC or stage III lung cancer. Given that the predicting the disease can include the accurate predicting of cancer stages, cancer stage-specific treatments can be provided, based on the accurate cancer stage-specific predictions. For example, high disease score-based predictions for early-stage cancers can inform first line treatments or resections, or more aggressive monitoring of the patient. In some examples, the predicted disease state comprises a prediction of the tissue of origin for the CRCF. That is, analyzing the CRCF can be used to predict the tissue from which the cfDNA fragment corresponding to the CRCF, originated. For example, based on features of the CRCF, it could be predicted that the cfDNA fragment may have originated, e.g., broken off, from genomic DNA of a liver cell.

Processes 100, 200, and 300 are identical, except that process 200 includes additional analyses when compared to process 100, as indicated in 202 and 204, and process 300 includes additional analyses when compared to process 200, as indicated in 302. Process 100, 200, or 300 can be performed, for example, using one or more electronic devices implementing a software platform. In some examples, process 100, 200, or 300 is performed using a client-server system, and the blocks of process 100, 200, or 300 are divided up in any manner between the server and a client device. In other examples, the blocks of process 100, 200, or 300 are divided up between the server and multiple client devices. Thus, while portions of process 100, 200 or 300 are described herein as being performed by particular devices of a client-server system, it will be appreciated that process 100, 200, or 300 is not so limited. In other examples, process 100, 200, or 300 is performed using only a client device or only multiple client devices. In process 100, 200, or 300, some blocks are, optionally, combined, the order of some blocks is, optionally, changed, and some blocks are, optionally, omitted. In some examples, additional steps may be performed in combination with the process 100, 200, or 300. Accordingly, the operations as illustrated (and described in greater detail below) are exemplary by nature and, as such, should not be viewed as limiting.

In some instances, the disclosed methods may be used to predict a disease by assessing a count of methylation loci in at least 1, at least 2, at least 3, at least 4, at least 5, at least 6, at least 7, at least 8, at least 9, at least 10, at least 20, at least 30, at least 40, or more than 40 gene loci.

In some instances, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAPI, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CRCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPKI, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CSI, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, or VEGFB gene locus, or any combination thereof.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a biopsy sample (e.g., a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom).

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

The disclosed methods may be used with any of a variety of samples. For example, in some instances, the sample may comprise a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the disclosed methods may further comprise analyzing a primary control (e.g., a normal liquid sample). In some instances, the disclosed methods may further comprise determining if a primary control is available and, if so, isolating a control nucleic acid (e.g., DNA) from said primary control. In some instances, the sample may comprise any normal control (e.g., a normal adjacent liquid (NAL)) if no primary control is available. In some instances, the sample may be or may comprise a histologically normal liquid sample. In some instances, the method includes evaluating a sample, e.g., a histologically normal liquid sample using the methods described herein. In some instances, the disclosed methods may further comprise acquiring a sub-sample enriched for non-tumor cells, e.g., by macro-separating a non-tumor liquid sample from said NAL in a sample not accompanied by a primary control. In some instances, the disclosed methods may further comprise determining that no primary control and no NAL is available, and marking said sample for analysis without a matched control.

In some instances, samples obtained from histologically normal liquid samples (e.g., otherwise histologically normal liquid margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of liquid samples (or disease states thereof), e.g., metastatic lesions, or liquid biopsy samples. Liquid samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample can comprise cell-free DNA (cfCDNA) and/or circulating tumor DNA (ctDNA). cfDNA is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, the cfDNA fragments extracted from a sample may comprise a mixture of tumor cfDNA fragments and non-tumor cfDNA fragments. In some instances, the tumor cfDNA fragments may be derived from a tumor portion of a heterogeneous liquid biopsy sample, and the non-tumor cfDNA fragments may be derived from a normal portion of the heterogeneous liquid biopsy sample. In some instances, the sample may comprise a liquid biopsy sample, and the tumor cfDNA fragments may be derived from a circulating tumor DNA (ctDNA) fraction of the liquid biopsy sample while the non-tumor cfDNA fragments may be derived from a non-tumor, cell-free DNA (cfDNA) fraction of the liquid biopsy sample.

In some instances, DNA is extracted from nucleated cells from the sample. In some instances, a sample may have a low nucleated cellularity, e.g., when the sample is comprised mainly of erythrocytes, lesional cells that contain excessive cytoplasm. In some instances, a sample with low nucleated cellularity may require more, e.g., greater, liquid volume for DNA extraction.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises cfDNA from a tumor or from a normal liquid sample. In certain instances, the sample may further comprise a non-nucleic acid component, e.g., cells, protein, carbohydrate, or lipid, e.g., from the tumor or normal liquid sample.

### Subjects

In some instances, the sample is obtained (e.g., collected) from a subject (e.g., patient) with a condition or disease (e.g., a hyperproliferative disease or a non-cancer indication) or suspected of having the condition or disease. The subject can provide a test sample or one or more reference samples, and the samples can be processed according to the methods described herein. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myclofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AmoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

cfDNA may be extracted from blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

One exemplary cfDNA extraction procedure, for example, comprises (i) collection of the fluid sample (e.g., whole blood) from which cfDNA is to be extracted from plasma in the blood. More specifically, the fluid sample may be treated with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

### Library Preparation

In some instances, the nucleic acids isolated from the sample may be used to construct a library (e.g., a nucleic acid library as described herein). In some instances, the nucleic acids are optionally subjected to repair of end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of cfDNA fragments. As described herein, the cfDNA fragments of the library can include a target cfDNA fragment (e.g., a tumor cfDNA fragment, a reference cfDNA fragment and/or a control cfDNA fragment; also referred to herein as a first, second and/or third cfDNA fragment, respectively). The cfDNA fragments of the library can be from a single subject or individual. In some instances, a library can comprise cfDNA fragments derived from more than one subject (e.g., 2, 3, 4, 5, 6, 7, 8, 9, 10, 20, 30 or more subjects). For example, two or more libraries from different subjects can be combined to form a library having cfDNA fragments from more than one subject (where the cfDNA fragments derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring cfDNA fragment, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor cfDNA fragment. In some instances, the subgenomic interval comprises a non-tumor cfDNA fragment.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a cfDNA fragment (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target cfDNA fragments to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of cfDNA fragments (e.g., a library catch) captured from one or more nucleic acid libraries. For example, the method may comprise: providing one or a plurality of nucleic acid libraries, each comprising a plurality of cfDNA fragments (e.g., a plurality of target cfDNA fragments and/or reference cfDNA fragments) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/cfDNA fragment hybrids; separating the plurality of target capture reagent/cfDNA fragment hybrids from said hybridization mixture, e.g., by contacting said hybridization mixture with a binding entity that allows for separation of said plurality of target capture reagent/cfDNA fragment hybrids from the hybridization mixture, thereby providing a library catch (e.g., a selected or enriched subgroup of cfDNA fragments from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11): 903-5; Hodges, E. et al. (2007) Nat. Genet. 39(12): 1522-7; and Okou, D. T. et al. (2007) Nat. Methods 4(11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual cfDNA fragments (e.g., as in single molecule sequencing) or clonally expanded proxies for individual cfDNA fragments in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, Pacific Biosciences' PacBio® RS platform, Ultima Genomics' UG100™ platform, or the Illumina's NovaSeq X series platform. In some instances, sequencing may comprise Illumina MiSeqÔ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor cfDNA fragments from a sample; (b) simultaneously or sequentially contacting the library with one, two, three, four, five, or more than five pluralities of target capture reagents under conditions that allow hybridization of the target capture reagents to the target cfDNA fragments, thereby providing a selected set of captured normal and/or tumor cfDNA fragments (i.e., a library catch); (c) separating the selected subset of the cfDNA fragments (e.g., the library catch) from the hybridization mixture, e.g., by contacting the hybridization mixture with a binding entity that allows for separation of the target capture reagent/cfDNA fragment hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; I aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981“, “Identification of Common Molecular Subsequen” es”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (197“) “A General Method Applicable to the Search for Similarities in the Amino Acid Sequence of Two Prote” ns”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a pull down, bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27 (11): 1571-1572).

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27 (11): 1571-1572), TARGOMICS (Garinet, et al. (2017), “Calling Chromosome Alterations, DNA Methylation Statuses, and Mutations in Tumors by Simple Targeted Next-Generation Sequenci-g—A Solution for Transferring Integrated Pangenomic Studies into Routine Practice?”, J. Molecular Diagnostics 19 (5): 776-787), Bicycle (Grana, et al. (2018) “Bicycle: A Bioinformatics Pipeline to Analyze Bisulfite Sequencing Data”, Bioinformatics 34 (8): 1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for predicting a disease based on analyzing cfDNA fragments in a sample from a subject. The systems may comprise, e.g., one or more processors, and a memory unit communicatively coupled to the one or more processors and configured to store instructions that, when executed by the one or more processors, cause the system to: determine, by one or more processors, a test count of methylated loci for a computationally reconstructed cfDNA fragment (CRCF) within a size range for a test sample obtained from a subject; determine, by the one or more processors, a disease score based on comparing the determined test count of the methylated loci to a reference count of methylated loci from one or more reference samples; and predict, by the one or more processors, a disease state for the subject based on a comparison of the disease score to a predetermined disease score threshold. The system can comprise further instructions, that when executed by the one or more processors, cause the system to: bin sizes of the CRCFs; and determine the size range based on the binned sizes. The system can comprise further instructions that, when executed by the one or more processors, cause the system to: preprocess one or more methylation statuses of one or more nucleotides from one or more ends of sequence reads from the CRCFs. The preprocessing can be based on one or more methylation statuses of other sequence reads from the CRCFs. The preprocessing can comprise computationally removing the one or more nucleotides from the one or more ends of the sequence reads. The preprocessing can comprise computationally correcting the one or more methylation statuses of the one or more nucleotides.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for predicting a diseased based on analyzing cfDNA fragments in any of a variety of samples as described herein (e.g., a tissue sample, biopsy sample, hematological sample, or liquid biopsy sample derived from the subject).

In some instances, the plurality of gene loci for which sequencing data is processed to determine a disease score may comprise at least 1, 2, 3, 4, 5, 6, 7, 8, 9, 10, 20, 30, 40, 50, 60, 70, 80, 0, 100, 200, 300, 400, 500, 600, 700, 800, 900, 1000, or more than 1000 gene loci (or any number of gene loci within the range of 1 to more than 1000 gene loci).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a disease score is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, ƒ, where ƒ may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

As described elsewhere in the present application, a CRCF can be assigned a flag that designates the CRCF as being significantly different, i.e., significantly altered, from the one or more reference samples, e.g., a reference probability distribution. For a test sample, the fraction of fragments that are significantly different, i.e., significantly altered, from the one or more reference samples can be determined, and optionally, visualized. The fraction of fragments that are significantly altered can be used as an input for a machine learning method, such as one or more of the machine learning methods described herein. The fraction of fragments that are significantly altered can be used, for example, to predict a tissue of origin for a test sample, based on analysis from the one or more machine learning methods described herein.

### Computer Systems and Networks

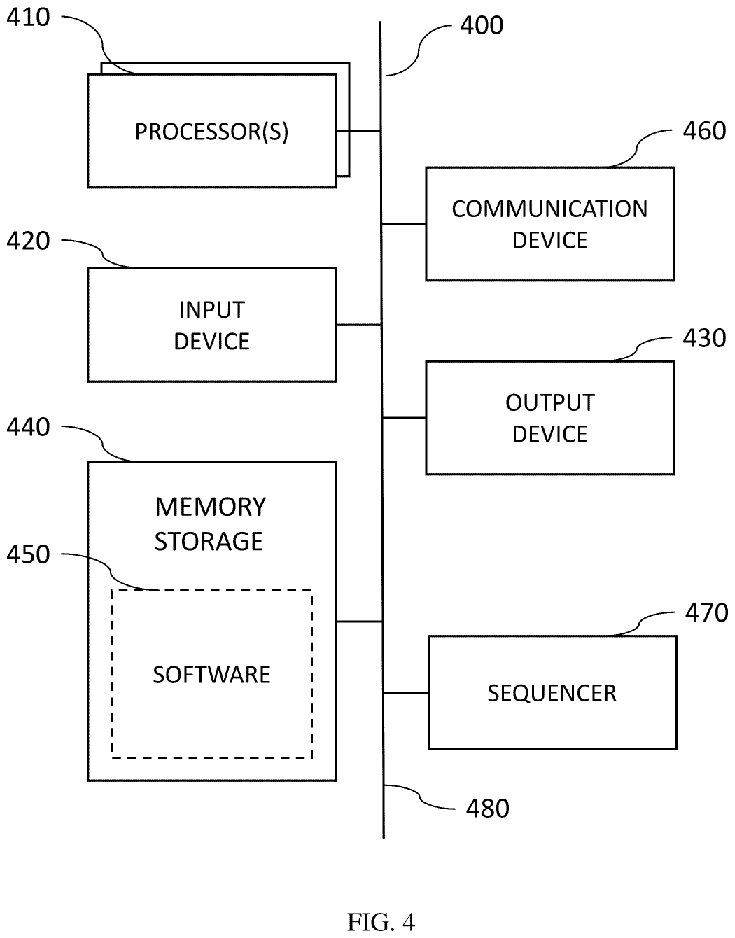

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Disease Prediction module 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

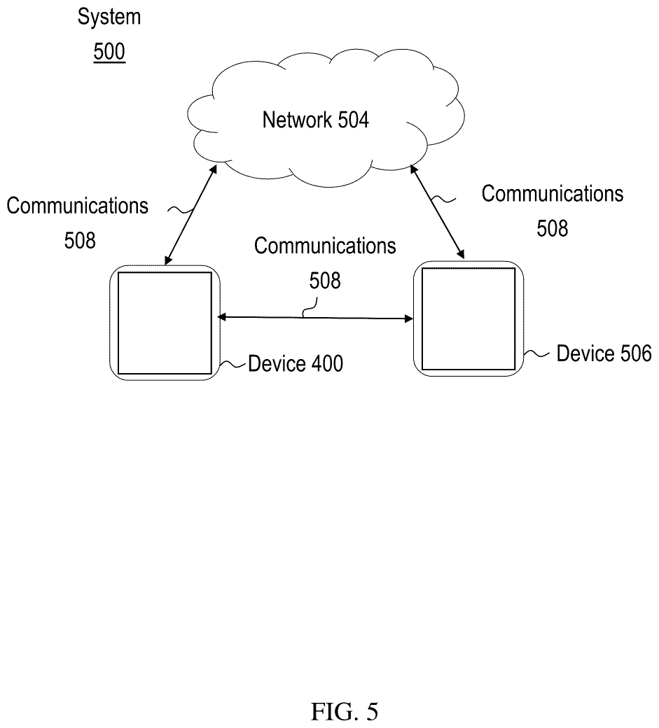

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 400 and 506 may communicate, e.g., using suitable communication interfaces via network 1004, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### Clinical Applications Based on the Predicting the Disease State

In some instances, the disclosed methods for predicting a disease based on cfDNA fragments may be used to diagnose (or as part of a diagnosis of) the presence of disease or other condition (e.g., cancer, genetic disorders (such as Down Syndrome and Fragile X), neurological disorders. In some instances, the disclosed methods may be applicable to diagnosis of any of a variety of cancers as described elsewhere herein.

In some instances, the disclosed methods for predicting a disease based on cfDNA fragments may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for predicting a disease based on cfDNA fragments may be used to select a subject (e.g., a patient) for a clinical trial based on the disease score value determined for one or more gene loci. In some instances, patient selection for clinical trials based on, e.g., identification of methylation at one or more gene loci, may accelerate the development of targeted therapies and improve the healthcare outcomes for treatment decisions.

In some instances, the disclosed methods for predicting a disease based on cfDNA fragments may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erlcada), asciminib hydrochloride (Scemblix), atczolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for predicting a disease based on cfDNA fragments may be used in treating a disease (e.g., a cancer) in a subject. For example, in response to determining a disease score using any of the methods disclosed herein, an effective amount of an anti-cancer therapy or anti-cancer treatment may be administered to the subject.

In some instances, the disclosed methods for predicting a disease based on cfDNA fragments may be used for monitoring disease progression or recurrence (e.g., cancer or tumor progression or recurrence) in a subject. For example, in some instances, the methods may be used to determine a disease score in a first sample obtained from the subject at a first time point, and used to determine a disease score in a test sample obtained from the subject at a second time point, where comparison of the first determination of the disease score and the second determination of the disease score allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the disease score.

In some instances, the value of the disease score determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for predicting a disease based on cfDNA fragments may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for predicting a disease based on cfDNA fragments as part of a genomic profiling process (or inclusion of the output from the disclosed methods for predicting a disease based on cfDNA fragments as part of the genomic profile of the subject) can improve the validity of, e.g., disease detection calls and treatment decisions, made on the basis of the genomic profile by, for example, independently confirming the presence of the disease in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some instances, the methods for predicting a disease state for the subject as described herein can be used to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

### EXEMPLARY EMBODIMENTS

Exemplary implementations of the methods and systems described herein include:

Embodiment 1. A method comprising:
providing a plurality of cfDNA fragments obtained from a sample from a subject;
ligating one or more adapters onto one or more cfDNA fragments from the plurality of cfDNA fragments;
amplifying the one or more ligated cfDNA fragments from the plurality of cfDNA fragments;
capturing amplified cfDNA fragments from the amplified cfDNA fragments;
sequencing, by a sequencer, the captured cfDNA fragments to obtain a plurality of sequence reads that represent the captured cfDNA fragments;
receiving, by one or more processors, sequence read data for the plurality of sequence reads;
aligning, by the one or more processors, the sequence read data to a reference genome, thereby generating computationally reconstructed cfDNA fragments (CRCFs);
determining, by the one or more processors, a test count of methylated loci for a CRCF of the CRCFs, within a size range, for a test sample obtained for the subject;
determining, by the one or more processors, a disease score based on comparing the test count of the methylated loci to a reference count of methylated loci from one or more reference samples; and
predicting, by the one or more processors, the disease state for the subject based on a comparison of the disease score to a predetermined disease score threshold.

Embodiment 2. The method of embodiment 1, further comprising:
binning, by the one or more processors, sizes of the CRCFs; and
determining, by the one or more processors, the size range based on the binned sizes.

Embodiment 3. The method of embodiment 1 or 2, further comprising:
preprocessing one or more methylation statuses of one or more nucleotides from one or more ends of sequence reads from the CRCFs.

Embodiment 4. The method of embodiment 3, wherein the preprocessing is based on one or more methylation statuses of other sequence reads from the CRCFs.

Embodiment 5. The method of embodiment 3 or 4, wherein the preprocessing comprises computationally removing the one or more nucleotides from the one or more ends of the sequence reads.

Embodiment 6. The method of any of embodiments 3-5, wherein the preprocessing comprises computationally correcting the one or more methylation statuses of the one or more nucleotides.

Embodiment 7. The method of any of embodiments 3-6, wherein the disease state indicates being at risk or having colorectal cancer (CRC).

Embodiment 8. The method of embodiment 7, wherein the colorectal cancer is stage 1 CRC, stage II CRC, stage III CRC, or stage IV CRC.

Embodiment 9. The method of any of embodiments 1-8, wherein the disease state indicates being at risk or having lung cancer.

Embodiment 10. The method of any of embodiments 1-9, wherein the lung cancer is stage I lung cancer, stage II lung cancer, stage III lung cancer, or stage IV lung cancer.

Embodiment 11. The method of any of embodiments 1-10, wherein the disease score is a probability from binomial testing, or a Kullback-Leibler divergence.

Embodiment 12. The method of embodiment 11, wherein the probability from the binomial testing is based on the determined test count of the methylated loci and the reference count of methylated loci.

Embodiment 13. The method of any of embodiments 1-12, wherein the subject is suspected of having or is determined to have cancer.

Embodiment 14. The method of embodiment 13, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Embodiment 15. The method of embodiment 13, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

Embodiment 16. The method of any of embodiments 1-15, further comprising obtaining the test sample from the subject.

Embodiment 17. The method of any of embodiments 1-16, wherein the test sample comprises a liquid biopsy sample, or a normal control.

Embodiment 18. The method of embodiment 17, wherein the test sample is a liquid biopsy sample and comprises blood, plasma, cerebrospinal fluid, sputum, stool, urine, or saliva.

Embodiment 19. The method of embodiment 18, wherein the test sample is a liquid biopsy sample and comprises circulating tumor cells (CTCs).

Embodiment 20. The method of embodiment 18, wherein the test sample is a liquid biopsy sample and comprises cell-free DNA (cfDNA).

Embodiment 21. The method of embodiment 20, wherein the cell-free DNA (cfDNA) or a portion thereof comprises circulating tumor DNA (ctDNA).

Embodiment 22. The method of any of embodiments 1-21, wherein the plurality of cfDNA fragments comprises a mixture of tumor cfDNA fragments and non-tumor cfDNA fragments.

Embodiment 23. The method of embodiment 22, wherein the tumor cfDNA fragments are derived from a tumor portion of a heterogeneous liquid biopsy sample, and the non-tumor cfDNA fragments are derived from a normal portion of the heterogeneous liquid biopsy sample.

Embodiment 24. The method of embodiment 23, wherein the test sample comprises a liquid biopsy sample, and wherein the tumor cfDNA fragments are derived from a circulating tumor DNA (ctDNA) fraction of the liquid biopsy sample, and the non-tumor cfDNA fragments are derived from a non-tumor, cell-free DNA (cfDNA) fraction of the liquid biopsy sample.

Embodiment 25. The method of any of embodiments 1-24, wherein the one or more adapters comprise amplification primers, flow cell adaptor sequences, substrate adapter sequences, or sample index sequences.

Embodiment 26. The method of any of embodiments 1-25, wherein the captured cfDNA fragments are captured from the amplified cfDNA fragments by hybridization to one or more bait molecules.

Embodiment 27. The method of embodiment 26, wherein the one or more bait molecules comprise one or more cfDNA fragments, each comprising a region that is complementary to a region of a captured cfDNA fragment.

Embodiment 28. The method of any of embodiments 1-27, wherein amplifying cfDNA fragments comprises performing a polymerase chain reaction (PCR) amplification technique, a non-PCR amplification technique, or an isothermal amplification technique.

Embodiment 29. The method of any of embodiments 1-28, wherein the sequencing comprises use of a massively parallel sequencing (MPS) technique, whole genome sequencing (WGS), whole exome sequencing, targeted sequencing, direct sequencing, or Sanger sequencing technique.

Embodiment 30. The method of embodiment 29, wherein the sequencing comprises massively parallel sequencing, and the massively parallel sequencing technique comprises next generation sequencing (NGS).

Embodiment 31. The method of any of embodiments 1-30, wherein the sequencer comprises a next generation sequencer.

Embodiment 32. The method of any of embodiments 1-31, wherein one or more of the plurality of sequencing reads overlap one or more gene loci within one or more subgenomic intervals in the sample.

Embodiment 33. The method of embodiment 32, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

Embodiment 34. The method of embodiment 32 or 33, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAPI, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CRCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPKI, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

Embodiment 35. The method of embodiment 32 or 33, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CSI, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.

Embodiment 36. The method of any of embodiments 1-35, further comprising generating, by the one or more processors, a report indicating the determined disease score.

Embodiment 37. The method of embodiment 36, further comprising transmitting the report to a healthcare provider.

Embodiment 38. The method of embodiment 36 or 37, wherein the report is transmitted via a computer network or a peer-to-peer connection.

Embodiment 39. A method of predicting a disease state of a subject, comprising:
determining, by one or more processors, a test count of methylated loci for a computationally reconstructed cfDNA fragment (CRCF) within a size range for a test sample obtained from a subject;
determining, by the one or more processors, a disease score based on comparing the test count of the methylated loci to a reference count of methylated loci from one or more reference samples; and
predicting, by the one or more processors, the disease state for the subject based on a comparison of the disease score to a predetermined disease score threshold.

Embodiment 40. The method of embodiment 39, further comprising:
binning, by the one or more processors, sizes of the CRCFs; and determining, by the one or more processors, the size range based on the binned sizes.

Embodiment 41. The method of embodiment 39 or 40, further comprising:
preprocessing one or more methylation statuses of one or more nucleotides from one or more ends of sequence reads from the CRCFs.

Embodiment 42. The method of embodiment 41, wherein the preprocessing is based on one or more methylation statuses of other sequence reads from the CRCFs.

Embodiment 43. The method of embodiment 41 or 42, wherein the preprocessing comprises computationally removing the one or more nucleotides from the one or more ends of the sequence reads.

Embodiment 44. The method of any of embodiments 41-43, wherein the preprocessing comprises computationally correcting the one or more methylation statuses of the one or more nucleotides.

Embodiment 45. The method of any of embodiments 39-44, wherein the disease state indicates being at risk or having colorectal cancer (CRC).

Embodiment 46. The method of embodiment 39, wherein the colorectal cancer is stage 1 CRC, stage II CRC, stage III CRC, or stage IV CRC.

Embodiment 47. The method of any of embodiments 39-46, wherein the disease state indicates being at risk or having lung cancer.

Embodiment 48. The method of any of embodiments 39-47, wherein the lung cancer is stage I lung cancer, stage II lung cancer, stage III lung cancer, or stage IV lung cancer.

Embodiment 49. The method of any of embodiments 39-48, wherein the predicted disease state comprises a prediction of the tissue of origin for the CRCF.

Embodiment 50. The method of any of embodiments 39-49, wherein the disease score is a probability from binomial testing or a Kullback-Leibler divergence.

Embodiment 51. The method of embodiment 50, wherein the probability from the binomial testing is based on the test count of the methylated loci and the reference count of methylated loci.

Embodiment 52. The method of embodiment 50 or 51, wherein the disease state is based on the probability from the binomial testing being less than the predetermined disease score threshold.

Embodiment 53. The method of any of embodiments 39-52, wherein the predetermined disease score threshold is 0.05.

Embodiment 54. The method of any of embodiments 39-53, wherein the Kullback-Leibler divergence is based on a test probability distribution of methylated loci counts and a reference probability distribution of methylated loci counts.

Embodiment 55. The method of any of embodiments 39-54, wherein the one or more reference samples comprise a healthy sample.

Embodiment 56. The method of any of embodiments 39-55, wherein the test sample is a disease sample.

Embodiment 57. The method of any of embodiments 39-56, wherein the one or more reference samples are obtained from a reference subject.

Embodiment 58. The method of any of embodiments 39-57, wherein the reference count of methylated loci is based on a synthetic reference set of sequence read count data.

Embodiment 59. The method of embodiment 58, wherein the synthetic reference set of sequence read count data is based on a composite profile.

Embodiment 60. The method of embodiment 59, wherein the composite profile is based on the one or more reference samples.

Embodiment 61. The method of any of embodiments 39-60, wherein the test sample is obtained from the subject.

Embodiment 62. The method of any of embodiments 39-61, wherein the test count of methylated loci is a normalized count.

Embodiment 63. The method of embodiment 62, wherein the normalized count is the test count of methylated loci normalized by a count of methylated and unmethylated loci.

Embodiment 64. The method of any of embodiments 40-63, the binning further comprising generating an empirical distribution function of the sizes of the CRCFs.

Embodiment 65. The method of embodiment 64, the determining the size range being based on the empirical distribution function.

Embodiment 66. The method of any of embodiments 40-65, the determining the size range being based on Otsu thresholding.

Embodiment 67. The method of any of embodiments 39-66, wherein the size range corresponds to a nucleosome position in a genome.

Embodiment 68. The method of embodiment 67, wherein the size range comprises a lower boundary of approximately 140 nucleotides in length and an upper boundary of approximately 200 nucleotides in length.

Embodiment 69. The method of embodiment 67 or 68, wherein the size range comprises a lower boundary of approximately 280 nucleotides in length and an upper boundary of approximately 400 nucleotides in length.

Embodiment 70. The method of any of embodiments 67-69, wherein the size range comprises a lower boundary of approximately 450 nucleotides in length and an upper boundary of approximately 600 nucleotides in length.

Embodiment 71. The method of any of embodiments 67-70, wherein the size range comprises a lower boundary of approximately 280 nucleotides in length and an upper boundary of approximately 600 nucleotides in length.

Embodiment 72. The method of any embodiments 39-71, wherein the determining the disease score is used to diagnose or confirm a diagnosis of the disease state in the subject.

Embodiment 73. The method of any of embodiments 39-72, wherein the disease state comprises having a cancer.

Embodiment 74. The method of embodiment 69 or 73, further comprising selecting an anti-cancer therapy to administer to the subject based on the determining of the disease score.

Embodiment 75. The method of any of embodiments 69-74, further comprising determining an effective amount of an anti-cancer therapy to administer to the subject based on the determining of the disease score.

Embodiment 76. The method of any of embodiments 69-75, further comprising administering an anti-cancer therapy to the subject based on the determining of the disease score.

Embodiment 77. The method of any of embodiments 69-76, wherein the anti-cancer therapy comprises chemotherapy, radiation therapy, immunotherapy, a targeted therapy, or surgery.

Embodiment 78. A method for monitoring cancer progression or recurrence in a subject, the method comprising:
determining a first disease score in a first test sample obtained from the subject at a first time point according to the method of any one of embodiments 1-77;
determining a second disease score in a second test sample obtained from the subject at a second time point; and
comparing the first test sample to the second test sample, thereby monitoring the cancer progression or recurrence.

Embodiment 79. The method of embodiment 78, wherein the second disease score for the second test sample is determined according to the method of any of embodiments 1-78.

Embodiment 80. The method of embodiment 78 or 79, further comprising selecting an anti-cancer therapy for the subject in response to the cancer progression.

Embodiment 81. The method of embodiment 78 or 79, further comprising administering an anti-cancer therapy to the subject in response to the cancer progression.

Embodiment 82. The method of embodiment 78 or 79, further comprising adjusting an anti-cancer therapy for the subject in response to the cancer progression.

Embodiment 83. The method of any one of embodiments 78-80, further comprising adjusting a dosage of the anti-cancer therapy or selecting a different anti-cancer therapy in response to the cancer progression.

Embodiment 84. The method of embodiment 83, further comprising administering the adjusted anti-cancer therapy to the subject.

Embodiment 85. The method of any one of embodiments 78-84, wherein the first time point is before the subject has been administered an anti-cancer therapy, and wherein the second time point is after the subject has been administered the anti-cancer therapy.

Embodiment 86. The method of any one of embodiments 78-85, wherein the subject has a cancer, is at risk of having a cancer, is being routine tested for cancer, or is suspected of having a cancer.

Embodiment 87. The method of any one of embodiments 78-86, wherein the cancer is a solid tumor.

Embodiment 88. The method of any one of embodiments 78-87, wherein the cancer is a hematological cancer.

Embodiment 89. The method of any one of embodiments 78-88, wherein the anti-cancer therapy comprises chemotherapy, radiation therapy, immunotherapy, a targeted therapy, or surgery.

Embodiment 90. The method of any one of embodiments 39-89, further comprising determining, identifying, or applying the predicted disease score for the sample as a diagnostic value associated with the test sample.

Embodiment 91. The method of any one of embodiments 39-90, further comprising generating a genomic profile for the subject based on the determining of the predicted disease score.

Embodiment 92. The method of embodiment 91, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 93. The method of embodiment 91 or 92, wherein the genomic profile for the subject further comprises results from a nucleic acid sequencing-based test.

Embodiment 94. The method of any one of embodiments 91-93, further comprising selecting an anti-cancer therapy, administering an anti-cancer therapy, or applying an anti-cancer therapy to the subject based on the generated genomic profile.

Embodiment 95. The method of any one of embodiments 39-94, wherein the determining of the predicted disease score for the test sample is used in making suggested treatment decisions for the subject.

Embodiment 96. The method of any one of embodiments 39-95, wherein the determining of the predicted disease score for the test sample is used in applying or administering a treatment to the subject.

Embodiment 97. A system comprising:
one or more processors; and
a memory communicatively coupled to the one or more processors and configured to store instructions that, when executed by the one or more processors, cause the system to:
   determine, by one or more processors, a test count of methylated loci for a computationally reconstructed cfDNA fragment (CRCF) within a size range for a test sample obtained from a subject;
   determine, by the one or more processors, a disease score based on comparing the determined test count of the methylated loci to a reference count of methylated loci from one or more reference samples; and
   predict, by the one or more processors, a disease state for the subject based on a comparison of the disease score to a predetermined disease score threshold.

Embodiment 98. The system of embodiment 97, comprising further instructions that, when executed by the one or more processors, cause the system to:
bin sizes of the CRCFs; and
determine the size range based on the binned sizes.

Embodiment 99. The system of embodiment 97 or 98, comprising further instructions that, when executed by the one or more processors, cause the system to:
preprocess one or more methylation statuses of one or more nucleotides from one or more ends of sequence reads from the CRCFs.

Embodiment 100. The system of embodiment 99, wherein the preprocessing is based on one or more methylation statuses of other sequence reads from the CRCFs.

Embodiment 101. The system of embodiment 99 or 100, wherein the preprocessing comprises computationally removing the one or more nucleotides from the one or more ends of the sequence reads.

Embodiment 102. The system of any of embodiments 99-101, wherein the preprocessing comprises computationally correcting the one or more methylation statuses of the one or more nucleotides.

Embodiment 103. A non-transitory computer-readable storage medium storing one or more programs, the one or more programs comprising instructions, which when executed by one or more processors of a system, cause the system to:
determine, by one or more processors, a test count of methylated loci for a computationally reconstructed cfDNA fragment (CRCF) within a size range for a test sample obtained from the subject;
determine, by the one or more processors, a disease score based on comparing the determined test count of the methylated loci to a reference count of methylated loci from one or more reference samples; and
predict, by the one or more processors, a disease state for the subject based on a comparison of the disease score to a predetermined disease score threshold.

Embodiment 104. The non-transitory computer-readable storage medium of embodiment 103, comprising further instructions that, when executed by the one or more processors of a system, cause the system to:
bin sizes of the CRCFs; and
determine the size range based on the binned sizes.

Embodiment 105. The non-transitory computer-readable storage medium of embodiment 103 or 104, comprising further instructions, that, when executed by the one or more processors of a system, cause the system to:
preprocess one or more methylation statuses of one or more nucleotides from one or more ends of sequence reads from the CRCFs.

Embodiment 106. The non-transitory computer-readable storage medium of embodiment 105, wherein the preprocessing is based on one or more methylation statuses of other sequence reads from the CRCFs.

Embodiment 107. The non-transitory computer-readable storage medium of embodiment 105 or 106, wherein the preprocessing comprises computationally removing the one or more nucleotides from the one or more ends of the sequence reads.

Embodiment 108. The non-transitory computer-readable storage medium of any of embodiments 105-107, wherein the preprocessing comprises computationally correcting the one or more methylation statuses of the one or more nucleotides.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

The following examples further demonstrate to one skilled in the art how to make and use the methods and systems described herein, and are not intended to limit the scope of the claimed invention.

### Example 1

This section provides an example of analyzing computationally reconstructed cfDNA fragments (CRCFs) for predicting whether samples are derived from a healthy sample or a tumor sample. The present Example stratified the CRCFs into three size ranges and assessed the ability to predict the tumor sample based on one of the three size ranges. The CRCFs analyzed for the present Example derived from many pooled healthy samples and many pooled tumor samples from multiple subjects. Subject DNA underwent DNA enzymatic methylation conversion chemistry and whole genome next generation sequencing (NGS) at a sequencing depth of 100×. The resulting raw reads were aligned to the human genome, and methylation statuses of CpG nucleotides and their loci in individual DNA molecules were determined using methods well known in the art. Each CRCF was then assigned to a CRCF size range and then within the size range, the molecule was analyzed via binomial testing to determine if the molecule was likely to have derived from a healthy sample or a tumor sample. If the p-value from the binomial significance testing was less than a predetermined disease score threshold of 0.05, the experimenter concluded that at least some number of the observed cfDNA molecules were like to have not come from a healthy subject, and the p-value was interpreted as a likelihood that the subject may have a tumor.

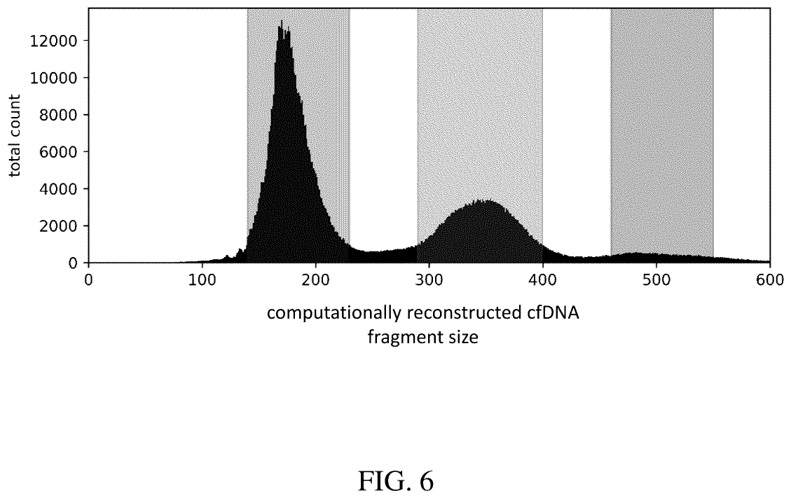

FIG. 6 provides a histogram of CRCF sizes. That is, counts of the CRCFs across a number of contiguous size bins are depicted. The x-axis depicts the range of CRCF sizes. The y-axis depicts the counts, i.e., frequency, for each bin of the CRCFs. The distribution of the CRCF sizes is multi-modal, and a rectangle outlines the bounds of each peak of the distribution. The bounds of each of the rectangles also defines the size range of the CRCFs to be analyzed downstream, e.g., the size range of CRCFs that are to be subject to methylation analysis. The smallest sized size range captures the sharpest peak of the distribution, and ranges from a lower bound of about 140 base pairs to an upper bound of about 220 base pairs. The intermediary sized size range captures an intermediately sharp peak of the distribution, and ranges from a lower bound of about 290 base pairs to an upper bound of about 400 base pairs. The largest size range captures the broadest peak of the distribution, and ranges from a lower bound of 450 base pairs to an upper bound of about 550 base pairs. A subset of the CRCFs for each size range were then analyzed regarding the molecules' methylation counts, and the results are depicted in the subsequent figures. The lower and upper bounds of each of the three size ranges depicted were determined via inspection.

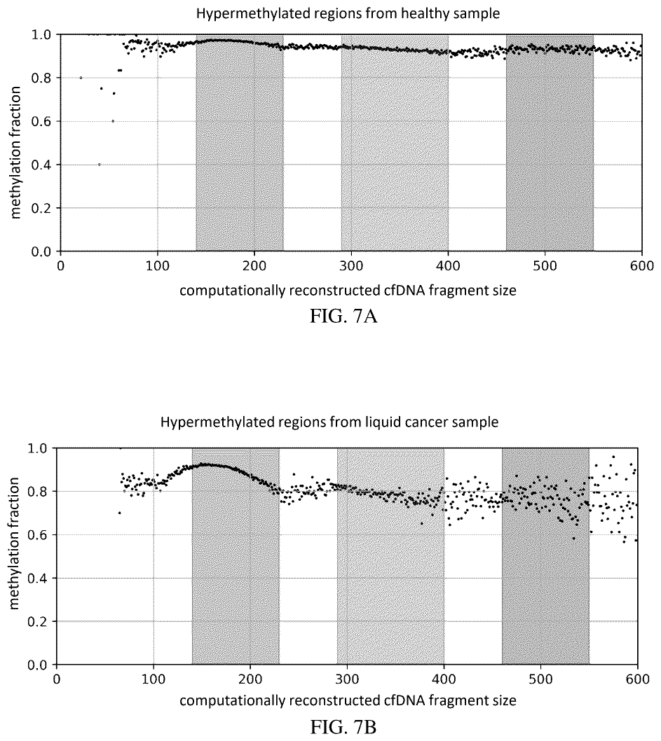

FIGS. 7A and 7B provide data depicting methylation fraction as a function of CRCF size. The size ranges depicted for both FIG. 7A and FIG. 7B correspond to the size ranges depicted in FIG. 6. For FIGS. 7A and 7B, the smallest size range has bounds ranging from about 140-220 base pairs, the intermediary size range has bounds ranging from about 290-400 base pairs, and the largest size range has bounds ranging from about 450-550 base pairs. FIG. 7A provides an example of data depicting methylation fraction as a function of cfDNA molecule size, for hypermethylated regions, from a healthy sample. FIG. 7B provides an example of data depicting methylation fraction as a function of CRCF size, for hypermethylated regions, from a cancer sample.

FIGS. 8A-8B provides data comparing healthy samples versus tumor samples for CRCFs of a specific size range, for regions that are typically hypomethylated in healthy samples. The left panel of FIG. 8A provides a histogram comparing 140-200 base pair long CRCFs for healthy versus tumor samples for sequences that are hypomethylated in healthy samples, using binomial testing. The x-axis depicts the fraction of altered fragments, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 8A provides a receiver-operating characteristic (ROC) curve for a binomial test-based classifier distinguishing the healthy versus tumor sample CRCFs shown in the left panel of FIG. 8A. As is convention for ROC curves, the x-axis depicts the false positive rate and the y-axis depicts the true positive rate, for the binomial testing's ability to distinguish the healthy samples from the tumor samples, at a given cutoff, when iterating across a range of cutoffs from 0 to 0.5. A cutoff comprising a desirable trade-off between the true positive rate and the false positive rate was selected at 0.008 and is indicated with the arrowhead in the right panel of FIG. 8A. Correspondingly, for the cutoff, a vertical line at x=0.008 that splits the healthy sample distribution from the tumor sample distribution for the left panel of FIG. 8A can be understood. From the ROC curve, the area under the curve (AUC) was determined at a value of 0.722. The left panel of FIG. 8B provides a histogram comparing 300-400 base pair long CRCFs for healthy versus tumor samples for sequences that are hypomethylated in healthy samples, using binomial testing. The x-axis depicts the fraction of altered CRCFs, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 8B provides an ROC curve for a binomial test-based classifier distinguishing the healthy versus tumor sample CRCFs shown in the left panel of FIG. 8B. As is convention for ROC curves, the x-axis depicts the false positive rate and the y-axis depicts the true positive rate, for the binomial testing's ability to distinguish the healthy samples from the tumor samples, at a given cutoff, when iterating across a range of cutoffs from 0 to 0.5. A cutoff comprising a desirable trade-off between the true positive rate and the false positive rate was selected at 0.011 and is indicated with the arrowhead in the right panel of FIG. 8B. Correspondingly, for the cutoff, a vertical line at x=0.011 that splits the healthy sample distribution from the tumor sample distribution for the left panel of FIG. 8B can be understood. From the ROC curve, the AUC was determined at a value of 0.775.

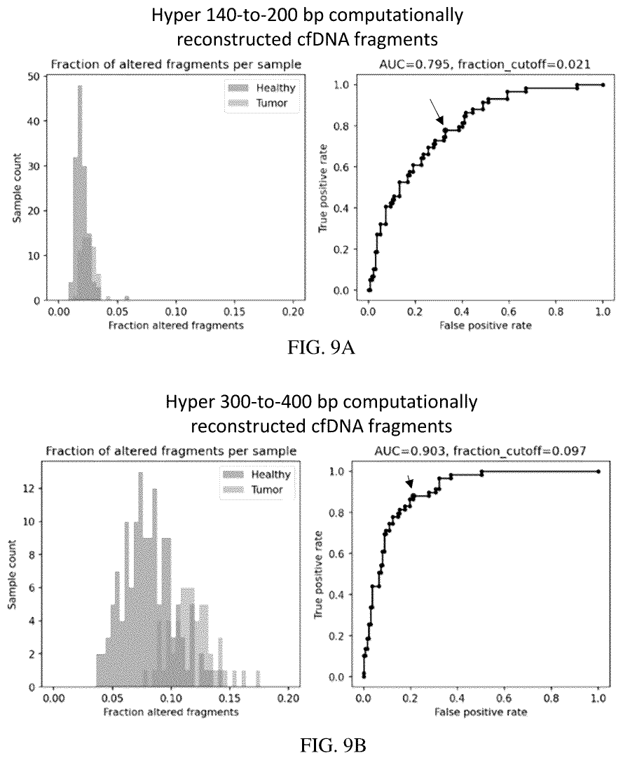

FIGS. 9A-9B provides data comparing healthy samples versus tumor samples for CRCFs of a specific size range, for regions that are typically hypermethylated in healthy samples. The left panel of FIG. 9A provides a histogram comparing 140-200 base pair long cfDNA molecules for healthy versus tumor samples for sequences that are hypermethylated in healthy samples, using binomial testing. The x-axis depicts the fraction of altered CRCFs, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 9A provides an ROC curve for a binomial test-based classifier distinguishing the healthy versus tumor sample CRCFs shown in the left panel of FIG. 9A. As is convention for ROC curves, the x-axis depicts the false positive rate and the y-axis depicts the true positive rate, for the binomial testing's ability to distinguish the healthy samples from the tumor samples, at a given cutoff, when iterating across a range of cutoffs from 0 to 0.5. A cutoff comprising a desirable trade-off between the true positive rate and the false positive rate was selected at 0.021 and is indicated with the arrowhead in the right panel of FIG. 9A. Correspondingly, for the cutoff, a vertical line at x=0.021 that splits the healthy sample distribution from the tumor sample distribution for the left panel of FIG. 9A can be understood. From the ROC curve, the AUC was determined at a value of 0.795.

The left panel of FIG. 9B provides a histogram comparing 300-400 base pair long CRCFs for healthy versus tumor samples for sequences that are hypermethylated in healthy samples, using binomial testing. The x-axis depicts the fraction of altered fragments, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 9B provides a ROC curve for a binomial test-based classifier distinguishing the healthy versus tumor sample cfDNA molecules shown in the right panel of FIG. 9B. As is convention for ROC curves, the x-axis depicts the false positive rate and the y-axis depicts the true positive rate, for the binomial testing's ability to distinguish the healthy samples from the tumor samples, at a given cutoff, when iterating across a range of cutoffs from 0 to 0.5. A cutoff comprising a desirable trade-off between the true positive rate and the false positive rate was selected at 0.097 and is indicated with the arrowhead in the right panel of FIG. 9B. Correspondingly, for the cutoff, a vertical line at x=0.097 that splits the healthy sample distribution from the tumor sample distribution for the left panel of FIG. 9B can be understood. From the ROC curve, the AUC was determined at a value of 0.903.

FIGS. 8A-9D together show that larger CRCFs ranging from 300-400 base pairs, are more effective at predicting tumors from samples, e.g., stage 1 plasma samples, 100×depth sequencing data. The AUC is higher when using larger CRCFs to separate the molecules that derive from healthy samples versus tumor samples, regardless of whether the cfDNA molecules map to hypomethylated or hypermethylated regions. Therefore, in some examples, larger cfDNA molecules, e.g., cfDNA molecules of 300-400 base pairs are most effective at predicting a disease.

### Example 2

This section provides an example of analyzing cfDNA molecules for predicting whether samples are derived from a healthy sample or a tumor sample. The present Example stratified the CRCFs into three size ranges and assessed the ability to predict the tumor sample based on one of the three size ranges. The CRCFs analyzed for the present Example derived from many pooled healthy samples and many pooled tumor samples from multiple subjects. Subject DNA underwent DNA methylation conversion chemistry and whole genome next generation sequencing (NGS) at a sequencing depth of 100×. The resulting raw reads were aligned to the human genome, and methylation statuses of CpG nucleotides and their loci in individual DNA molecules were determined using methods well known in the art. Each CRCF was then assigned to a CRCF size range and then within the size range, the CRCF was analyzed via binomial testing to determine if the CRCF was likely to have derived from a healthy sample or a tumor sample. If the p-value from the binomial significance testing was less than a predetermined disease score threshold of 0.05, the experimenter concluded that at least some number of the observed cfDNA were like to have not come from a healthy subject, and the p-value was interpreted as a likelihood that the subject may have a tumor. The present Example compares predictions based on CRCFs of a select size range, e.g., smaller-sized CRCFs or larger-sized CRCFs, against all CRCFs. Unlike Example 1, the present Example analyzes two size ranges of CRCFs, as opposed to three size ranges of CRCFs.

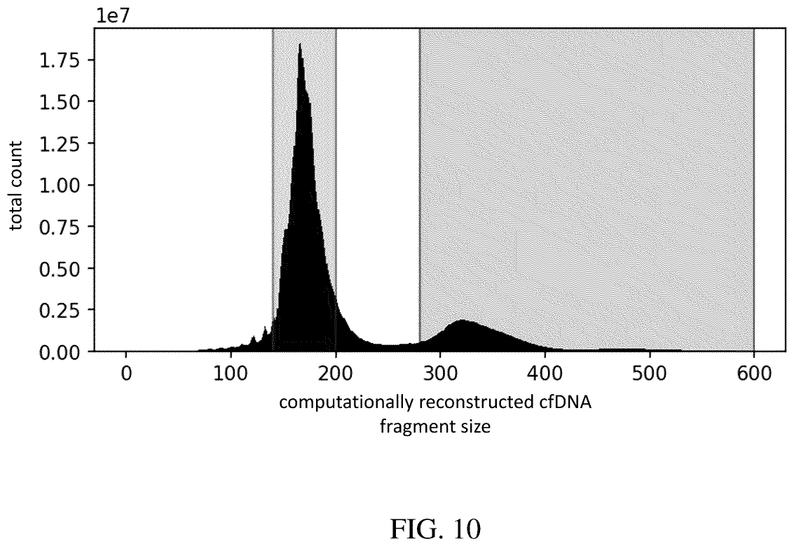

FIG. 10 provides a histogram of CRCF sizes. That is, counts of the CRCFs across a number of contiguous size bins are depicted. The x-axis depicts the range of CRCF sizes. The y-axis depicts the counts, i.e., frequency, for each bin of the CRCFs by magnitudes of 1e7. The distribution of the CRCF sizes is multi-modal, and a rectangle outlines the bounds of each peak of the distribution. The bounds of each of the rectangles also defines the size range of the CRCFs to be analyzed downstream, e.g., the size range of CRCFs that are to be subject to methylation analysis. The smallest sized size range captures the sharpest peak of the distribution, and ranges from a lower bound of about 140 base pairs to an upper bound of about 200 base pairs. The largest size range captures the broadest peak of the distribution, and ranges from a lower bound of 280 base pairs to an upper bound of about 600 base pairs. A subset of the CRCFs for each size range were then analyzed regarding the CRCFs' methylation counts, and the results are depicted in the subsequent figures. The lower and upper bounds of each of the two size ranges depicted were determined via inspection.

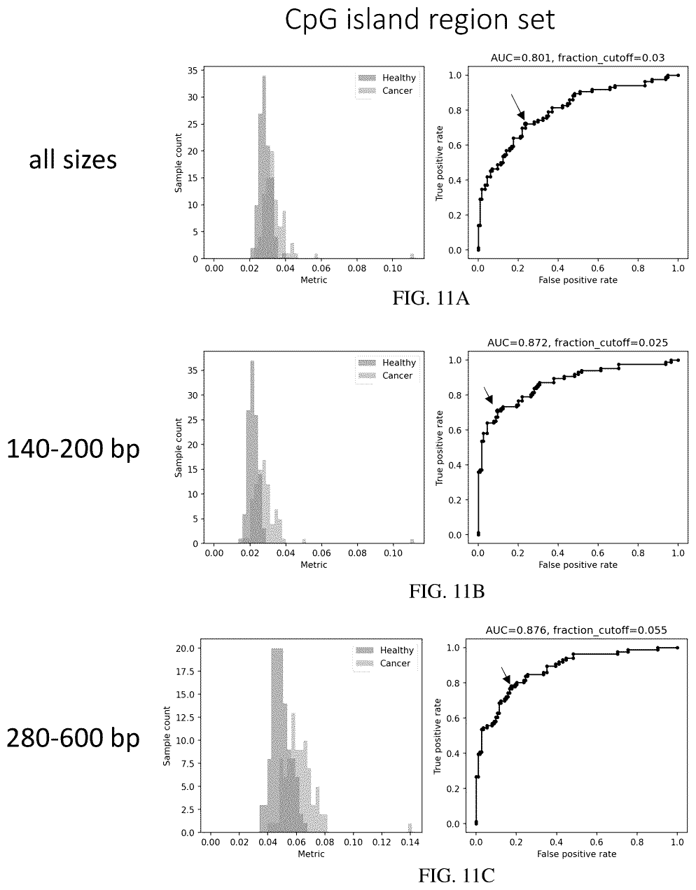

FIGS. 11A-13C provide data depicting methylation fraction as a function of cfDNA molecule size. The size ranges depicted for FIGS. 11A-13C correspond to the size ranges depicted in FIG. 10. For FIGS. 11A-13C, the smallest size range has bounds ranging from about 140-220 base pairs, and the largest size range has bounds ranging from about 280-600 base pairs. Example data for all CRCF sizes are also provided.

FIGS. 11A-C provide data comparing CRCFs for healthy versus cancer samples for sequences mapping to CpG islands, using binomial testing. The left panel of FIG. 11A provides an example histogram comparing CRCFs of all sizes for healthy versus cancer samples for sequences mapping to CpG islands, using binomial testing. The x-axis depicts the fraction of altered CRCFs, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 11A provides a ROC curve for a binomial test-based classifier distinguishing the healthy versus cancer sample CRCFs shown in the left panel of FIG. 11A. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.03 and is indicated with the arrowhead in the right panel of FIG. 11A. Correspondingly, for the cutoff, a vertical line at x=0.03 that splits the healthy sample distribution from the cancer sample distribution for the left panel of FIG. 11A can be understood. From the ROC curve, the area under the curve (AUC) was determined at a value of 0.801.

The left panel of FIG. 11B provides a histogram comparing cfDNA molecules for 140-200 base pair long cfDNA molecules for healthy versus cancer samples for sequences mapping to CpG islands, using binomial testing. The x-axis depicts the fraction of altered fragments, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 11B provides an ROC curve for a binomial test-based classifier distinguishing the healthy versus cancer sample CRCFs shown in the left panel of FIG. 11B. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.025 and is indicated with the arrowhead in the right panel of FIG. 11B. Correspondingly, for the cutoff, a vertical line at x=0.025 that splits the healthy sample distribution from the cancer sample distribution for the left panel of FIG. 11B can be understood. From the ROC curve, the area under the curve (AUC) was determined at a value of 0.872.

The left panel of FIG. 11C provides a histogram comparing CRCFs for 200 base pair and longer CRCFs for healthy versus cancer samples for sequences mapping to CpG islands, using binomial testing. The x-axis depicts the fraction of altered CRCFs, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 11C provides an example ROC curve for a binomial test-based classifier distinguishing the healthy versus cancer sample cfDNA molecules shown in the left panel of FIG. 11C. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.055 and is indicated with the arrowhead in the right panel of FIG. 11C. Correspondingly, for the cutoff, a vertical line at x=0.055 that splits the healthy sample distribution from the cancer sample distribution for the left panel of FIG. 11C can be understood. From the ROC curve, the area under the curve (AUC) was determined at a value of 0.876.

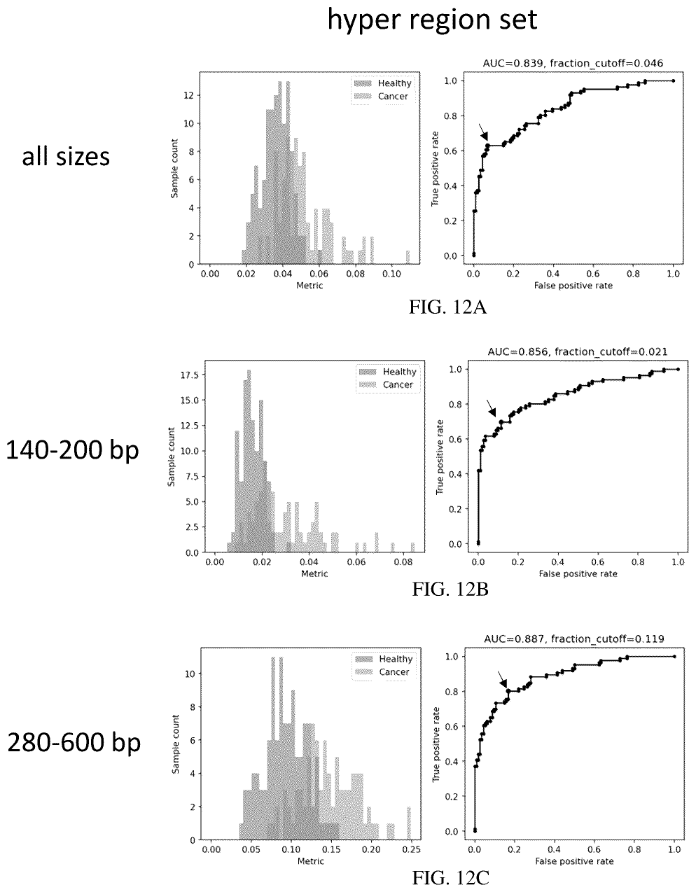

FIGS. 12A-12C provide data comparing cfDNA molecules for healthy versus cancer samples for sequences hypermethylated in healthy samples, using binomial testing. The left panel of FIG. 12A provides a histogram comparing CRCFs of all sizes for healthy versus cancer samples for sequences hypermethylated in healthy samples, using binomial testing. The x-axis depicts the fraction of altered fragments, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 12A provides an example ROC curve for a binomial test-based classifier distinguishing the healthy versus cancer sample cfDNA molecules shown in the left panel of FIG. 12A. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.046 and is indicated with the arrowhead in the right panel of FIG. 12A. Correspondingly, for the cutoff, a vertical line at x=0.046 that splits the healthy sample distribution from the cancer sample distribution for the left panel of FIG. 12A can be understood. From the ROC curve, the area under the curve (AUC) was determined at a value of 0.839.

The left panel of FIG. 12B provides a histogram comparing CRCFs for 140-200 base pair long CRCFs for healthy versus cancer samples for sequences hypermethylated in healthy samples, using binomial testing. The x-axis depicts the fraction of altered fragments, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 12C provides an example ROC curve for a binomial test-based classifier distinguishing the healthy versus cancer sample CRCFs shown in the left panel of FIG. 12C. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.021 and is indicated with the arrowhead in the right panel of FIG. 12C. Correspondingly, for the cutoff, a vertical line at x=0.021 that splits the healthy sample distribution from the tumor sample distribution for the left panel of FIG. 12C can be understood. From the ROC curve, the area under the curve (AUC) was determined at a value of 0.839.

The left panel of FIG. 12C provides a histogram comparing cfDNA molecules for 200 base pair and longer cfDNA molecules for healthy versus cancer samples for sequences hypermethylated in healthy samples, using binomial testing. The x-axis depicts the fraction of altered CRCFs, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 12C provides a ROC curve for a binomial test-based classifier distinguishing the healthy versus cancer sample cfDNA molecules shown in the left panel of FIG. 12C. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.119 and is indicated with the arrowhead in the left panel of FIG. 12C. Correspondingly, for the cutoff, a vertical line at x=0.119 that splits the healthy sample distribution from the cancer sample distribution for the left panel of FIG. 12C can be understood. From the ROC curve, the area under the curve (AUC) was determined at a value of 0.887.

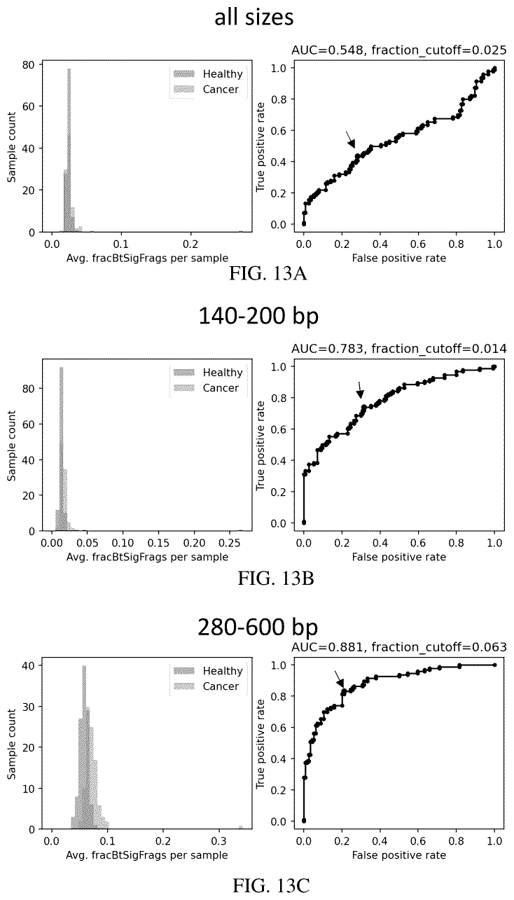

FIGS. 13A-13C provide data comparing CRCFs for healthy plasma versus cancer plasma samples, for sequences mapping to all informative regions, for subjects with stage 1 colorectal cancer, using binomial testing. The informative regions comprise approximately 39 000 intervals. The left panel of FIG. 13A provides a histogram comparing CRCFs of all sizes for healthy versus cancer samples for all informative sequences, using binomial testing. The x-axis depicts the average fraction of altered CRCFs, which refers to the fraction of CRCFs that are significantly different, i.e., significantly altered, from the one or more reference samples, for a given test sample, after performing the binomial testing. The y-axis depicts the count of samples for each bin of the fraction of altered CRCFs for the histogram bins. The right panel of FIG. 13A provides a ROC curve for a binomial test-based classifier distinguishing the healthy versus cancer sample CRCFs shown in the left panel of FIG. 13A. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.025 and is indicated with the arrowhead in the right panel of FIG. 13A. Correspondingly, for the cutoff, a vertical line at x=0.025 that splits the healthy sample distribution from the tumor sample distribution for the left panel of FIG. 13A can be understood. From the ROC curve, the area under the curve (AUC) was determined at a value of 0.025.

The left panel of FIG. 13B provides a histogram comparing CRCFs for 140-200 base pair long CRCFs for healthy versus cancer samples for all informative sequences in healthy samples, using binomial testing. The x-axis depicts the average fraction of altered fragments, which refers to the fraction of CRCFs that are significantly different, i.e., significantly altered, from the one or more reference samples, for a given test sample, after performing the binomial testing. The y-axis depicts the count of samples for each bin of the fraction of altered fragments for the histogram bins. The right panel of FIG. 13B provides an example ROC curve for a binomial test-based classifier distinguishing the healthy versus cancer sample CRCFs shown in the left panel of FIG. 13B. As is convention for ROC curves, the x-axis depicts the false positive rate and the y-axis depicts the true positive rate, for the binomial testing's ability to distinguish the healthy samples from the cancer samples, at a given cutoff, when iterating across a range of cutoffs from 0 to 0.5. A cutoff comprising a desirable trade-off between the true positive rate and the false positive rate was selected at 0.014 and is indicated with the arrowhead in the right panel of FIG. 13B. Correspondingly, for the cutoff, a vertical line at x=0.014 that splits the healthy sample distribution from the tumor sample distribution for the left panel of FIG. 13B can be understood. From the ROC curve, the area under the curve (AUC) was determined at a value of 0.014.

The left panel of FIG. 13C provides a histogram comparing CRCFs for 200 base pair and longer CRCFs for healthy versus cancer samples for all informative sequences, using binomial testing. The x-axis depicts the average fraction of altered fragments, which refers to the fraction of CRCFs that are significantly different, i.e., significantly altered, from the one or more reference samples, for a given test sample, after performing the binomial testing. The y-axis depicts the count of samples for each bin of the fraction of altered fragments for the histogram bins. The right panel of FIG. 13C provides a ROC curve for a binomial test-based classifier distinguishing the healthy versus cancer sample CRCFs shown in the left panel of FIG. 13C. As is convention for ROC curves, the x-axis depicts the false positive rate and the y-axis depicts the true positive rate, for the binomial testing's ability to distinguish the healthy samples from the tumor samples, at a given cutoff, when iterating across a range of cutoffs from 0 to 0.5. A cutoff comprising a desirable trade-off between the true positive rate and the false positive rate was selected at 0.063 and is indicated with the arrowhead in the right panel of FIG. 13C. Correspondingly, for the cutoff, a vertical line at x=0.063 that splits the healthy sample distribution from the cancer sample distribution for the left panel of FIG. 13C can be understood. From the ROC curve, the area under the curve (AUC) was determined at a value of 0.881.

For each of FIGS. 11A-13C, using only a single size range of CRCFs resulted in an improved ability to distinguish cfDNA molecules from healthy samples versus cancer samples. Improvements were seen regardless of whether only the 140-200 base pair size range was analyzed, or only the 280-600 base pair size range was analyzed. By comparison, analyzing all CRCF sizes resulted in poor separation between the healthy samples and the cancer samples. Therefore, improved disease predictions are achieved by analyzing a specific size range of cfDNA molecules.

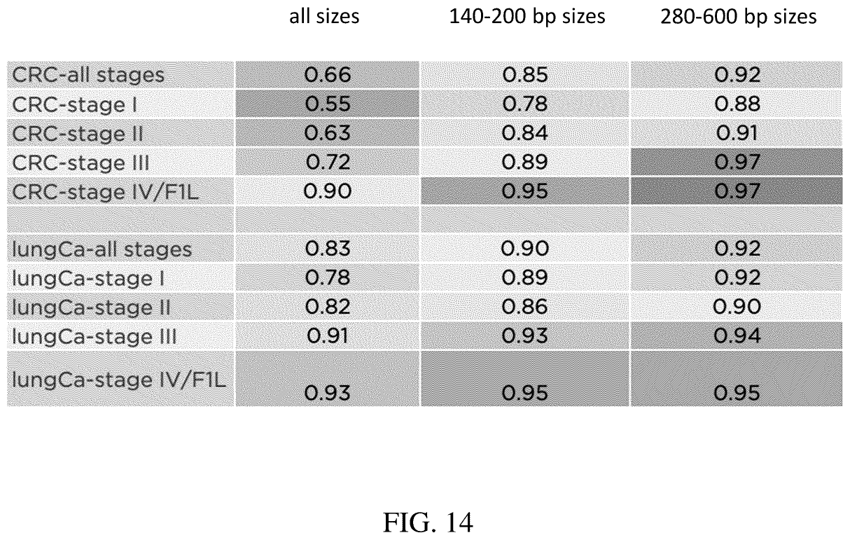

FIG. 14 provides heat-mapped tabular data for CRCFs of different size ranges and how predictive those different size ranges are for predicting CRC or lung cancer stages. The table values provide area under the curve (AUC) values, from a receiver-operating characteristic (ROC) analysis. The AUC values indicate the predictiveness of methylation counts of cfDNA molecules for a specific size range, including for all sizes. For both subjects with CRC cancers or lung cancers, at all stages, predicting the cancer based on all cfDNA molecules provides poor predictions, ranging from scores of 0.66 to 0.93. In contrast, for every single cancer stage and for the cancer overall, for both the CRC and the lung cancer, analyzing only the 140-200 base pair cfDNA size range, or only the 280-600 base pair cfDNA size range, resulted in improved predictions. More specifically, for this example, the 280-600 base pair cfDNA size range provided the best predictions, for all CRC and lung cancer stages.

1. A method comprising:
providing a plurality of cfDNA fragments obtained from a test sample from a subject; ligating one or more adapters onto one or more cfDNA fragments from the plurality of cfDNA fragments;
amplifying the one or more ligated cfDNA fragments from the plurality of cfDNA fragments;
capturing amplified cfDNA fragments from the amplified cfDNA fragments;
sequencing, by a sequencer, the captured cfDNA fragments to obtain a plurality of sequence reads that represent the captured cfDNA fragments;
receiving, by one or more processors, sequence read data for the plurality of sequence reads;
aligning, by the one or more processors, the sequence read data to a reference genome, thereby generating computationally reconstructed cfDNA fragments (CRCFs);
determining, by the one or more processors, a test count of methylated loci for a CRCF of the CRCFs, within a size range, for a test sample obtained for the subject;
determining, by the one or more processors, a disease score based on comparing the test count of the methylated loci to a reference count of methylated loci from one or more reference samples; and
predicting, by the one or more processors, the disease state for the subject based on a comparison of the disease score to a predetermined disease score threshold.

2-38. (canceled)

39. A method of predicting a disease state of a subject, comprising:
determining, by one or more processors, a test count of methylated loci for a computationally reconstructed cfDNA fragment (CRCF) within a size range for a test sample obtained from a subject;
determining, by the one or more processors, a disease score based on comparing the test count of the methylated loci to a reference count of methylated loci from one or more reference samples; and
predicting, by the one or more processors, the disease state for the subject based on a comparison of the disease score to a predetermined disease score threshold.

40. The method of claim 39, further comprising:
binning, by the one or more processors, sizes of the CRCFs; and
determining, by the one or more processors, the size range based on the binned sizes.

41. The method of claim 39, further comprising:
preprocessing one or more methylation statuses of one or more nucleotides from one or more ends of sequence reads from the CRCFs.

42. The method of claim 41, wherein the preprocessing is based on one or more methylation statuses of other sequence reads from the CRCFs.

43. The method of claim 41, wherein the preprocessing comprises computationally removing the one or more nucleotides from the one or more ends of the sequence reads.

44. The method of claim 41, wherein the preprocessing comprises computationally correcting the one or more methylation statuses of the one or more nucleotides.

45. The method of claim 39, wherein the disease state indicates being at risk or having colorectal cancer (CRC) or lung cancer.

46-48. (canceled)

49. The method of claim 39, wherein the predicted disease state comprises a prediction of the tissue of origin for the CRCF.

50. The method of claim 39, wherein the disease score is a probability from binomial testing or a Kullback-Leibler divergence.

51-57. (canceled)

58. The method of claim 39, wherein the reference count of methylated loci is based on a synthetic reference set of sequence read count data.

59-61. (canceled)

62. The method of claim 39, wherein the test count of methylated loci is a normalized count.

63. The method of claim 62, wherein the normalized count is the test count of methylated loci normalized by a count of methylated and unmethylated loci.

64. The method of claim 40, the binning further comprising generating an empirical distribution function of the sizes of the CRCFs.

65-66. (canceled)

67. The method of claim 39, wherein the size range corresponds to a nucleosome position in a genome.

68. The method of claim 67, wherein the size range comprises
(a) a lower boundary of approximately 140 nucleotides in length and an upper boundary of approximately 200 nucleotides in length;
(b) a lower boundary of approximately 280 nucleotides in length and an upper boundary of approximately 400 nucleotides in length;
(c) a lower boundary of approximately 450 nucleotides in length and an upper boundary of approximately 600 nucleotides in length; or
(d) a lower boundary of approximately 280 nucleotides in length and an upper boundary of approximately 600 nucleotides in length.

69-73. (canceled)

74. The method of claim 39, further comprising selecting an anti-cancer therapy to administer to the subject based on the determining of the disease score.

75. The method of claim 39, further comprising determining an effective amount of an anti-cancer therapy to administer to the subject based on the determining of the disease score.

76. The method of claim 39, further comprising administering an anti-cancer therapy to the subject based on the determining of the disease score.

77-96. (canceled)

97. A system comprising:
one or more processors; and
a memory communicatively coupled to the one or more processors and configured to store instructions that, when executed by the one or more processors, cause the system to:
determine, by one or more processors, a test count of methylated loci for a computationally reconstructed cfDNA fragment (CRCF) within a size range for a test sample obtained from a subject;
determine, by the one or more processors, a disease score based on comparing the determined test count of the methylated loci to a reference count of methylated loci from one or more reference samples; and
predict, by the one or more processors, a disease state for the subject based on a comparison of the disease score to a predetermined disease score threshold.

98-108. (canceled)
